(19) United States
(12) Patent Application Publication
(10) Pub. No.: US 2025/0354133 A1
(43) Pub. Date: Nov. 20, 2025

(54) SIALIDASE FUSION MOLECULES AND RELATED USES

(71) Applicant: The Scripps Research Institute, La Jolla, CA (US)

(72) Inventors: Peng WU, San Diego, CA (US); Zhuo YANG, San Diego, CA (US); Ke QIN, San Diego, CA (US)

(21) Appl. No.: 18/862,182

(22) PCT Filed: May 4, 2023
(86) PCT No.: PCT/US2023/066586

§ 371 Date: Nov. 1, 2024

Related U.S. Application Data
(60) Provisional application No. 63/338,134, filed on May 4, 2022.
(60) Provisional application No. 63/480,228, filed on Jan. 17, 2023.

(51) Int. Cl. C12N 9/24; A61K 39/00; A61P 35/00; C07K 16/28; C07K 16/30

(52) U.S. Cl. CPC C12N 9/2402; A61P 35/00; C07K 16/2809; C07K 16/2863; C07K 16/2896; C07K 16/3015; A61K 2039/505; C07K 2317/31; C07K 2317/622; C07K 2319/30; C12Y 302/01018

(57) ABSTRACT

The invention provides fusion molecules that contain a sialidase and a bispecific molecule that engages an immune cell (e.g., T cell or NK cell) and a target cell underlying a disease. Related polynucleotides, vectors and host cells are also described herein. The invention further provides therapeutic applications of the sialidase fusions in treating cancer.

### CROSS-REFERENCE TO RELATED APPLICATIONS

The subject patent application claims the benefit of priority to U.S. Provisional Patent Application No. 63/480,228 (filed Jan. 17, 2023; now expired) and U.S. Provisional Patent Application No. 63/338,134 (filed May 4, 2022; now expired). The full disclosures of the priority applications are incorporated herein by reference in their entirety and for all purposes.

### STATEMENT OF GOVERNMENT SUPPORT

This invention was made with government support under contract numbers AI154138 and AI143884 awarded by the National Institutes of Health. The government has certain rights in the invention.

### SEQUENCE LISTING

This application incorporates by reference a Sequence Listing in the form of a ST.26 XML file labeled “2143_2US_Sequence Listing”. The file is of 54 KB in size and was created on Aug. 16, 2023.

### BACKGROUND OF THE INVENTION

A central theme in cancer immunotherapy is to activate patients' own immune system for tumor control. Bispecific T cell engagers (BiTEs) are off-the-shelf immunotherapy agents that recruit endogenous CD8+ and CD4+ T cells to eradicate tumor cells in a major histocompatibility complex (MHC)-independent manner. A BiTE molecule consists of two single-chain variable fragments (scFvs), one targets a tumor-associated antigen and the other binds to CD3 on T cells. These two scFvs are covalently connected by a small linker peptide. Blinatumomab targeting CD19 antigen present on B cells is the first BiTE approved by the US Food and Drug Administration (FDA) to treat B-cell precursor acute lymphoblastic leukemia (ALL) in patients who still have detectable traces of cancer after chemotherapy.

Like most T cell-based therapies, however, the promise of BiTEs for treating solid tumors is largely plagued by limited penetration into tumor tissue and immunosuppressive tumor microenvironments where suppression of T cells is orchestrated by the activity of tumor cells and the neighboring stromal myeloid and lymphoid cells. In this unique microenvironment alterations of cell-surface epitopes of tumor cells and immune cells take place as a result of limited availability of nutrients and accumulated metabolic waste products, which subsequently alters the interactions of tumor cells and tumor-infiltrating T cells (TILs) and ultimately leads to T cell exhaustion and poor tumor control. Therefore, enabling approaches that target the molecular and cellular components of the immunosuppressive tumor microenvironment may transform T cell-based cancer treatments, including those enabled by BiTEs.

There is a need in the art for means for enhancing effectiveness of immunotherapies based on bispecific immune cell engaging molecules such as BiTEs. The instant invention is directed to addressing these and other unmet needs.

### SUMMARY OF THE INVENTION

In one aspect, the invention provides fusion proteins that contain (a) a bispecific molecule or bispecific antibody and (b) a sialidase or enzymatic fragment thereof. The bispecific molecule in the fusion proteins contain two antibody fragments or moieties that respectively bind to an immune cell and an antigen associated with or implicated in a disease. In some embodiments, the bispecific molecule contains in tandem a first scFv targeting the immune cell and a second scFv targeting the disease antigen. In some of these embodiments, the bispecific antibody is a bispecific T cell engager (BiTE), and the first scFv recognizes a T cell-specific molecule. In some of these fusion proteins, the targeted T cell specific molecule is CD3. Some BiTE molecules employed in the BiTE-sialidase fusion proteins of the invention selectively engage γδT cells (e.g., Vγ9Vδ2 T cells). In these embodiments, the targeted T cell specific molecule is TCR on the cells (e.g., Vγ9Vδ2 TCR). In some other embodiments, the bispecific antibody is a bispecific innate cell engager, and the first scFv recognizes a surface antigen on an innate immune cell. In some of these embodiments, the targeted innate immune cell is NK cell or macrophage. In some of these embodiments, the surface antigen on the innate immune cell is CD16A or NKp44. Some sialidase fusion proteins of the invention target tumors. In these fusions, the second antibody fragment specifically binds to a tumor antigen. For example, some fusion proteins of the invention contain a bispecific molecule that engages the immune cell with a tumor cell expressing HER2 or PSMA.

In various embodiments, the sialidase in the fusion proteins of the invention is a human sialidase, a viral sialidase or a bacterial sialidase. In some embodiments, the fusion proteins employ human sialidase NEU1, NEU2, NEU3, NEU4 or isoform thereof. In some embodiments, the fusion proteins contain bacterial sialidase, for example, human commensal bacterium Bifidobacterium longum subspecies infantis (B. infantis) sialidase. In the fusion proteins, the sialidase can be fused either at the C-terminus or the N-terminus of the bispecific molecule. In some embodiments, the sialidase is fused to the bispecific molecule via a GS linker. In some of these embodiments, the employed GS linker can contain an amino acid sequence (GmS)n, wherein m is an integer from 1 to 6, and n is an integer from 1 to 10. As specific examples, the employed linker can be GGGSGGGS (SEQ ID NO:2), GGGGSGGGGS (SEQ ID NO:29), GGGGSGGGGSGGGGS (SEQ ID NO:30), or GGGGSGGGGSGGGSGGGS (SEQ ID NO:31). In some fusion proteins of the invention, the two antibody fragments or moieties (e.g., scFvs) are also connected by a GS linker.

In some fusion proteins of the invention, the employed bispecific molecule contains an amino acid sequence that is at least 95% or 99% identical to the sequence set forth in any one of SEQ ID NOs: 6, 10, 12, 14, 31, 32 and 40. In some embodiments, the bispecific molecule contains an amino acid sequence that is set forth in any one of SEQ ID NOs: 6, 10, 12, 14, 31, 32 and 40, or a conservatively modified variant thereof. Some of the sialidase fusion polypeptides of the invention contain an amino acid sequence that is at least 95% or 99% identical to the sequence set forth in any one of SEQ ID NOs: 7, 8, 11, 13, 15, 23-28 and 41. In some embodiments, the sialidase fusion protein contains an amino acid sequence that is set forth in any one of SEQ ID NOs: 7, 8, 11, 13, 15, 23-28 and 41, or a conservatively modified variant thereof.

In some other aspects, the invention provides polynucleotide molecules or sequences that encode the sialidase fusion proteins or polypeptides described herein. Related vectors and host cells that harbor such polynucleotide sequences are also encompassed by the invention. In some related aspects, the invention provides pharmaceutical compositions that contain a therapeutically effective amount of a sialidase fusion protein or an encoding polynucleotide sequence described herein, and a pharmaceutically acceptable carrier. Some polynucleotide sequences of the invention are directed to mRNAs. Some of these embodiments of the invention are directed to lipid nanoparticles (LNPs) that are formulated with one mRNA molecule described herein. Therapeutic combinations or kits containing the sialidase fusion proteins or polynucleotides are also provided in the invention.

In another aspect, the invention provides methods for treating or ameliorating the symptoms of a disease or disorder in a subject. The methods involve administering to the subject a pharmaceutical composition that contains a sialidase fusion polypeptide of the invention. Some methods of the invention are specifically directed to treating tumors.

A further understanding of the nature and advantages of the present invention may be realized by reference to the remaining portions of the specification and claims.

### DESCRIPTION OF THE DRAWINGS

FIG. 1: Sialic acid removal enhanced BiTE-induced T cell cytotoxicity and activation (a) Killing of HER2-positive SK-BR-3 cells induced by HER2-targeting 4D5 BiTEs with or without prior sialidase treatment. The effector: target cell ratio used for experiments in FIG. 1 is 5:1 (b) Killing of SK-BR-3 cells induced by 4D5 BiTEs+hPBMCs with or without prior treatment with 100 uM sialylation inhibitor P-3FAX-Neu5Ac. (c) Killing of SK-BR-3 cells and MCF7 cells induced by 4D5 BiTEs with or without prior sialidase treatment. (d) Killing of PSMA positive PC3 cells induced by PSMA targeting BiTEs with or without prior sialidase treatment. (e) CD25 and CD69 expression level was measured in T cells with or without the presence of 4D5 BiTEs and sialidase. (f) IFN-Y release was measured as an indicator of BiTE-induced T cell activation using MCF7 cells incubated with hPBMCs with or without the addition of sialidase. Mean values show three independent experiments with standard error of the mean (SEM) as error bars. For statistical analysis, unpaired Student t test with Welch correction was applied (*P<0.05, **P<0.01, ***P<0.001 and ****P<0.0001).

FIG. 2: Desialylation promotes stronger BITE-mediated immune synapse (IS) formation rather than suppressing the inhibitory Siglec signaling. (a) Siglec-7 and -9 expression levels were measured on human T cells with or without BiTE-induced activation, and with or without sialidase treatment. (b) Siglec-7 and -9 expression levels were measured on T cells and CD3 negative cells in PBMCs from different human donors. (c) SK-BR-3 cell killing induced by 4D5 BiTEs was measured with or without the addition of sialidase or anti-siglec-9 antibody. (d) Saturated recombinant CTLA-4 was added with sialidase to determine if the increased killing associated with sialidase treatment can be blocked. (e) Anti-CD2 blocking antibody was added to test the effects on the desialylation boosted killing (f) Staining of CD3, and actin to visualize the immune synapses formed by T cells and tumor cells by confocal microscopy. Two groups, with and without sialidase treatment of the tumor cells, were imaged. Scale bar=10 um. (g) CD3 accumulation at the IS was calculated by dividing the mean fluorescence intensity (MFI) at the IS by the MFI of the rest of the membrane. (h) Relative IS contact area was calculated by dividing the area of the IS by the area of the rest of the T cell membrane. All analysis was done using ImageJ. Mean values show three independent experiments with standard error of the mean (SEM) as error bars. For statistical analysis, unpaired Student t test with Welch correction was applied (*P<0.05, **P<0.01, ***P<0.001 and ****P<0.0001).

FIG. 3: Construction of 4D5 BiTE-sialidase fusion proteins for selective desialylation of HER2 positive cells. (a) Two fusion proteins are constructed by conjugating sialidase to either the N or C terminus of the 4D5 BiTE. (b) Measuring sialic acid levels on the surface of SK-BR-3 and SKOV-3 cells after the treatment of the fusion proteins and staining with FITC-SNA. Mean fluorescent intensity (MFI) is displayed on the figure. (c) HER2 positive SKOV-3 cells and HER2 negative MDA-MB-468 cells were mixed and treated with 5 nM or 50 nM 4D5 BiTE-sialidase. The cell-surface sialylation level was measured by FITC-SNA staining and flow cytometry analysis.

FIG. 4: 4D5 BiTE-sialidase fusion protein exhibits better activities than 4D5 BiTE alone for HER2 positive target cell killing and T cell activation. (a) and (b) The specific lysis of HER2 positive SK-BR-3 cells and SKOV-3 cells with 4 nM 4D5 BiTE or sialidase fusion proteins at the effector: target ratio of 5:1. (c) & (d) Dose dependent targeted killing using 4D5 BiTE or 4D5 BiTE-sialidase against SK-BR-3 and SKOV-3 cells. (e), (f) & (g) CD25, CD69 and CD107a expression level was measured in T cell populations in the presence of SK-BR-3 cells and 4 nM 4D5 BiTE or the fusion protein. (h), (i) & (j) IFN-γ, IL-2 and TNF-α release were measured for 4D5 BiTE- or fusion protein-induced T cell activation in the presence of SK-BR-3 cells. (k) Volcano plot of differentiated expressed genes between T cells treated with 4D5 BiTE or 4D5 BiTE-sialidase upon co-culturing with target MDA-MB-231 cells (genes with p value of fold changes <0.01 were displayed). (1) The cytotoxicity enhancements induced by 4D5 BiTE-sialidase as compared to 4D5 BiTE for cell lines with different HER2 expression levels. (m) The specific lysis of MDA-MB-468 cells under 4 nM 4D5 BiTE or 4D5 BiTE-sialidase at the effector: target ratio of 5:1. IC50 values were calculated from sigmoidal dose-response curve model using PRISM8. For statistical analysis, unpaired Student t test with Welch correction was applied (*P<0.05, **P<0.01, ***P<0.001 and ****P<0.0001).

FIG. 5: CD19 BiTE-sialidase exhibits superior activities than CD19 BiTE to induce in vitro tumor cell killing and T cell activation. (a) Dose-dependent killing of Raji cells induced by CD19 BiTE or CD19 BiTE-sialidase at the effector: target ratio of 10:1. (b), (c), (d) & (e) CD25, CD69, and CD107a expression levels were measured in T cell populations by flow cytometry analysis. (f), (g) & (h) IFN-γ, IL-2 and TNF-α release was measured for CD19 BiTE- or fusion protein-induced T cell activation in the presence of Raji cells. IC50 values were calculated from sigmoidal dose-response curve model using PRISM8. For statistical analysis, unpaired Student t test with Welch correction was applied (*P<0.05, **P<0.01, ***P<0.001 and ****P<0.0001).

FIG. 6: BiTE-sialidase fusion proteins exhibit better tumor control in vivo than BiTE. (a) Experimental timeline and treatment protocol for a HER2 positive SK-BR-3 breast cancer xenograft in NCG mice (n=5). (b) Serum IFN-γ release was measured 5 hours after the first drug treatment. (c) Bioluminescence was measured twice a week to visualize changes in tumor volume. On day 41, one mouse in the PBS control group died. (d) & (e) Bioluminescence was measured and calculated for each mouse as an indication of tumor burden. Tumor progression was followed by plotting change in the group average (d) values over time. (e) Experimental timeline and treatment protocol for a xenograft NALM-6 model of acute lymphoblastic leukemia. (f) Bioluminescence measured on day 3 and day 7 were shown for different groups for comparing the tumor burden. (g) Tumor burden was measured and calculated by tracking the bioluminescence signals (n=5). One-way ANOVA and student t test were used to analyze the differences among groups (*P<0.05, **P<0.01, ***P<0.001 and ****P<0.0001).

FIG. 7: EGFR BiTE-sialidase shows better tumor control by altering tumor immune cell compositions in a syngeneic mouse model of melanoma. (a) Measuring sialic acid levels on the surface of B16-E5 cells after the treatment of EGFR BiTE-sialidase with sialic acid binding lectin SNA. Mean fluorescent intensity (MFI) is displayed on the figure. (b) Tumor growth curve and percentage of survival. (c) of B16-E5 mice model under intratumor treatment of PBS, EGFR BiTE and EGFR BITE-sialidase. 0.6 million B16-E5 cells were initially inoculated in C57BL/6J mice and 0.5 mg BiTE and 0.93 mg BiTE-sialidase were given intratumorally on Day 8, 12 and 14, n=5 for each group.

FIG. 8: Comparison of desialylation efficiency of BiTE sialidase fusions and free sialidase. The desialylation efficiency was compared for three different constructs: 4D5 BiTE-sialidase, 4D5 sialidase-BiTE and free sialidase. Different concentrations of three constructs were used with SK-BR-3 cells in DMEM medium for 1 hour before the desialylation was measured by two lectins peanut agglutinin (PNA) and Maackia Amurensis Lectin II (MAL II).

FIG. 9: Structures of sialidase fused BiKE molecules.

FIG. 10: Comparison of NK cell mediated cytotoxicity promoted by BiKE and different design of BiKE-Sialidase fusion proteins.

FIG. 11: Representative flow cytometry dot plots showing selective desilylation activity of BiKE-CD19-Sia and BIKE-EGFR-Sia.

FIG. 12: BiKE-sialidase fusion proteins exhibit better tumor control in vivo than BiKE.

FIG. 13: Fraction of sialylated (SNA+) cells quantified by flow cytometry gating after treatment with various concentrations of sialidase fused anti-EGFR BiKE molecules. A549 cells were treated with various concentrations of BiKE-EGFR, anti-EGFR TAL4S, anti-EGFR SL4TA, and sialidase for 1h in serum-free medium, followed by staining with SNA-FITC in HBSS buffer for 30 min. Mean values show three independent experiments with standard error of the mean (SEM) as error bars.

FIG. 14: Profiling the efficacy of EGFR BiTE-sialidase in B16-E5 mice melanoma model. (a) Circulating T cells from mice treated with CD4 or CD8 depleting antibody were analyzed by flow cytometry. Representative data from a CD4 depleted mouse and a CD8 depleted mouse were shown on the figure. In the figure, data from two separate mice were shown and overlapped in one dot plot (the sample from each mouse was analyzed independently by the flow cytometry). Peripheral blood from each mouse was collected and was stained with anti-CD3, -CD4 and -CD8 antibody. The figure showed CD4 and CD8 staining in CD3 positive populations. (b) Tumor volume was monitored in groups of mice with CD4 or CD8 depletion or no depletion under the treatment of the EGFR BiTE-sialidase. Control group with PBS treatment was also included (n=5). CD44, CD61L (c) Tim3 and TCF1 (d) were stained among CD8 T cell group from tumors collected from different groups to profile CD8 T cell functionality. For statistical analysis, unpaired Student t test with Welch correction was applied (*P<0.05, **P<0.01, ***P<0.001 and ****P<0.0001).

### DETAILED DESCRIPTION

### I. Overview

Immunotherapies based on bispecific molecules that engage immune cells and target cells (e.g., BiTEs) to activate patients' immune system have gained momentum with the recent FDA approval of Blinatumomab for treating B cell malignancies. However, limited success has been achieved for targeting solid tumors. The present invention is predicated in part on the studies undertaken by the inventors to develop fusion proteins containing a sialidase and a bispecific immune cell engager (e.g., BiTE), which enhances tumor cell susceptibility to bispecific molecule-mediated killing. The sialidase fused bispecific molecules developed and examined by the inventors include BiTEs, as well as bispecific innate cell engagers such as bispecific killer cell engagers (BiKEs). As detailed herein, the inventors observed that BiTE-sialidase fusion molecules specifically remove sialoglycans at T cell-target tumor cell interface to boost the T cell-dependent tumor cell cytolysis. It was demonstrated that the enhanced tumor cell cytolysis is independent of the inhibitory sialoglycan-Siglec signaling, but due to stronger immunological synapse formation induced by BiTEs. As exemplifications, it was shown that BiTE-sialidase fusion proteins that target human epidermal growth factor receptor 2 (Her2) and CD19 exhibit remarkably better efficacy of killing tumor cells than the BiTE alone both in vitro and in vivo in a xenograft tumor models. Enhanced cytolysis activities were also observed with sialidase-BiTE fusions that target other tumor antigens, e.g., PSMA. Utilizing a syngeneic mouse model of melanoma, additional studies conducted by the inventors demonstrated that BiTE-sialidase fusion proteins have therapeutic advantages over the parent BiTE.

In further studies, the inventors observed selective desialylation by sialidase fused BIKEs targeting CD19 or EGFR. These sialidase fused BiKEs also showed enhanced cytotoxicity relative to free NK cells. In vivo efficacy of the sialidase fused BIKEs was also demonstrated with an EGFR-targeting BiKE-sialidase fusion protein in a syngeneic mouse model. These results indicate that the sialidase bispecific molecule fusions described herein (e.g., BiTE-sialidase fusions and BiKE-sialidase fusions) can be employed as the next generation bispecific immune cell engaging molecules for cancer immunotherapy.

In accordance with these studies, the invention provides fusion proteins containing a sialidase that is conjugated to a bispecific molecule or bispecific antibody that engages an immune cell (e.g., T cell or NK cell) and a target antigen associated with an disease or disorder (e.g., cancer). Related polynucleotide sequences, expression vectors and host cells, as well as their therapeutic applications are also encompassed by the invention.

The invention can employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Sambrook et al, ed. (1989) Molecular Cloning A Laboratory Manual (2nd ed.; Cold Spring Harbor Laboratory Press); Sambrook et al, ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al, eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al, eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described can be followed as in Current Protocols in Immunology, John Wiley & Sons, New York; Stites et al, eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al, eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al, (Elsevier, Amsterdam); Goldsby et al, eds. (2000) Kuby Immunology (4th ed.; W.H. Freeman & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlag); Sambrook and Russell (2001) Molecular Cloning: A Laboratory Manual (Cold Spring Harbor Press); Lewin (2003) Genes VIII (Prentice Hall, 2003); Harlow and Lane (1988) Antibodies: A Laboratory Manual (Cold Spring Harbor Press); Dieffenbach and Dveksler (2003) PCR Primer (Cold Spring Harbor Press).

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “antibody” or “antigen-binding fragment” refers to polypeptide chain(s) which exhibit a strong monovalent, bivalent or polyvalent binding to a given antigen, epitope or epitopes. Unless otherwise noted, antibodies or antigen-binding fragments used in the invention can have sequences derived from any vertebrate, camelid, avian or pisces species. They can be generated using any suitable technology, e.g., hybridoma technology, ribosome display, phage display, gene shuffling libraries, semi-synthetic or fully synthetic libraries or combinations thereof. Unless otherwise noted, the term “antibody” as used in the present invention includes intact antibodies, antigen-binding polypeptide fragments and other designer antibodies that are described below or well known in the art (see, e.g., Serafini, J Nucl. Med. 34:533-6, 1993).

An intact “antibody” typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Each heavy chain of an antibody is comprised of a heavy chain variable region (V_{H}) and a heavy chain constant region. The heavy chain constant region is comprised of three domains, C_{H1}, C_{H2} and C_{H3}. Each light chain is comprised of a light chain variable region (V_{L}) and a light chain constant region. The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system and the first component (Clq) of the classical complement system.

The V_{H} and V_{L} regions of an antibody can be further subdivided into regions of hypervariability, also termed complementarity determining regions (CDRs), which are interspersed with the more conserved framework regions (FRs). Each V_{H} and V_{L} is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined by, e.g., Kabat et al., Sequences of Proteins of Immunological Interest, U.S. Department of Health and Human Services, U.S. Government Printing Office (1987 and 1991).

Antibody fragments or antigen-binding fragments contain the antigen-binding portions of an intact antibody that retain capacity to bind the cognate antigen. Examples of such antibody fragments include (i) a Fab fragment, a monovalent fragment consisting of the V_{L}, V_{H}, C_{L} and CHI domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an intact antibody; (v) disulfide stabilized Fvs (dsFvs) which have an interchain disulfide bond engineered between structurally conserved framework regions; (vi) a single domain antibody (dAb) which consists of a V_{H} domain (see, e.g., Ward et al., Nature 341:544-546, 1989); and (vii) an isolated complementarity determining region (CDR).

In some preferred embodiments, antibodies employed for practicing the present invention are single chain antibodies. The term “single chain antibody” refers to a polypeptide comprising a V_{H} domain and a V_{L} domain in polypeptide linkage, generally linked via a spacer peptide, and which may comprise additional domains or amino acid sequences at the amino- and/or carboxyl-termini. For example, a single-chain antibody may comprise a tether segment for linking to the encoding polynucleotide. As an example, a single chain variable region fragment (scFv) is a single-chain antibody. Compared to the V_{L} and V_{H} domains of the Fv fragment which are coded for by separate genes, a scFv has the two domains joined (e.g., via recombinant methods) by a synthetic linker. This enables them to be made as a single protein chain in which the V_{L} and V_{H} regions pair to form monovalent molecules.

Antibodies or antigen-binding fragments for practicing the invention can be produced by enzymatic or chemical modifications of the intact antibodies, or synthesized de novo using recombinant DNA methodologies, or identified using phage display libraries. Methods for generating these antibodies or antigen-binding molecules are all well known in the art. For example, single chain antibodies can be identified using phage display libraries or ribosome display libraries, gene shuffled libraries (see, e.g., McCafferty et al., Nature 348:552-554, 1990; and U.S. Pat. No. 4,946,778). In particular, scFv antibodies can be obtained using methods described in, e.g., Bird et al., Science 242:423-426, 1988; and Huston et al., Proc. Natl. Acad. Sci. USA 85:5879-5883, 1988. Fv antibody fragments can be generated as described in Skerra and Plückthun, Science 240:1038-41, 1988. Disulfide-stabilized Fv fragments (dsFvs) can be made using methods described in, e.g., Reiter et al., Int. J. Cancer 67:113-23, 1996. Similarly, single domain antibodies (dAbs) can be produced by a variety of methods described in, e.g., Ward et al., Nature 341:544-546, 1989; and Cai and Garen, Proc. Natl. Acad. Sci. USA 93:6280-85, 1996. Camelid single domain antibodies can be produced using methods well known in the art, e.g., Dumoulin et al., Nature Struct. Biol. 11:500-515, 2002; Ghahroudi et al., FEBS Letters 414:521-526, 1997; and Bond et al., J Mol Biol. 332:643-55, 2003. Other types of antigen-binding fragments (e.g., Fab, F(ab′) 2 or Fd fragments) can also be readily produced with routinely practiced immunology methods. See, e.g., Harlow & Lane, Using Antibodies, A Laboratory Manual, Cold Spring Harbor Laboratory Press, Cold Spring Harbor, New York, 1998. In some preferred embodiments, scFv fragments used in the sialidase fusions of the invention can be produced via recombinant expression.

Bispecific T cell engager antibodies, bispecific T cell engager molecules, or simply bispecific T cell engagers (BiTEs) are used interchangeably herein and refer to a group of bispecific antibodies that contain in tandem two single chain variable fragments (scFv). One of the scFvs has binding specificity for the T cell receptor (TCR) complex, and the other recognizes an antigen on a target cell (e.g., a cell surface marker that is associated with or implicated in a disease.

A “fusion” protein or polypeptide refers to a polypeptide comprised of at least two polypeptides and a linking sequence or a linkage to operatively link the two polypeptides into one continuous polypeptide. The two polypeptides linked in a fusion polypeptide are typically derived from two independent sources, and therefore a fusion polypeptide comprises two linked polypeptides not normally found linked in nature.

“Linkage” refers to means of operably or functionally connecting two biomolecules (e.g., polypeptides or polynucleotides encoding two polypeptides), including, without limitation, recombinant fusion, covalent bonding, disulfide bonding, ionic bonding, hydrogen bonding, and electrostatic bonding. “Fused” refers to linkage by covalent bonding. A “linker” or “spacer” refers to a molecule or group of molecules that connects two biomolecules, and serves to place the two molecules in a preferred configuration with minimal steric hindrance. Various linkages can be used in the construction of the fusion molecules of the invention. In some preferred embodiments, the polypeptide components of the sialidase fusion proteins of the invention are linked by a peptide bond.

The term “operably linked” when referring to a nucleic acid, means a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame, in the generation of a fusion protein.

The term “polynucleotide” or “nucleic acid” as used herein refers to a polymeric form of nucleotides of any length, either ribonucleotides or deoxyribonucleotides, that comprise purine and pyrimidine bases, or other natural, chemically or biochemically modified, non-natural, or derivatized nucleotide bases. Polynucleotides of the embodiments of the invention include sequences of deoxyribopolynucleotide (DNA), ribopolynucleotide (RNA), or DNA copies of ribopolynucleotide (cDNA) which may be isolated from natural sources, recombinantly produced, or artificially synthesized. A further example of a polynucleotide is polyamide polynucleotide (PNA). The polynucleotides and nucleic acids may exist as single-stranded or double-stranded. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The polymers made of nucleotides such as nucleic acids, polynucleotides and polynucleotides may also be referred to herein as nucleotide polymers.

Polypeptides are polymer chains comprised of amino acid residue monomers which are joined together through amide bonds (peptide bonds). The amino acids may be the L-optical isomer or the D-optical isomer. In general, polypeptides refer to long polymers of amino acid residues, e.g., those consisting of at least more than 10, 20, 50, 100, 200, 500, or more amino acid residue monomers. However, unless otherwise noted, the term polypeptide as used herein also encompass short peptides which typically contain two or more amino acid monomers, but usually not more than 10, 15, or 20 amino acid monomers.

Proteins are long polymers of amino acids linked via peptide bonds and which may be composed of two or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, and antibodies. In some embodiments, the terms polypeptide and protein may be used interchangeably.

The enzyme sialidase or neuraminidase was first isolated from the bacterium Vibrio cholerae. This enzyme specifically cleaves the terminal sialic acid moieties from sialomucins and glycoproteins. The loss of PAS or alcian blue staining following sialidase treatment is clearly indicative of the presence of sialic acid in tissue specimens. If the combined alcian blue-PAS protocol is performed following sialidase treatment, sialomucins that normally would stain blue with alcian blue stain red with PAS. Other than bacterial sialidases, enzymes with similar activities have also been identified from viral species (e.g., influenza viruses) and mammals (e.g., human).

As used herein, the term “target molecule” or “target antigen” refers to a molecule of interest on the surface of a target cell (e.g., tumor cell) that is to be specifically recognized by the bispecific molecule in the sialidase fusion proteins of the invention. Preferably, the target molecule for practicing the present invention is a polypeptide (e.g., a cellular receptor or surface marker protein).

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

A “conservative substitution” with respect to proteins or polypeptides refers to replacement of one amino acid with another amino acid having a similar side chain. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Methods of identifying nucleotide and amino acid conservative substitutions which do not eliminate protein activity are well-known in the art (see, e.g., Brummell et ah, Biochem. 32:1180-1 187 (1993); Kobayashi et ah, Protein Eng. 12 (10): 879-884 (1999); and Burks et al, Proc. Natl. Acad. Sci. USA 94:412-417 (1997)).

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Methods of alignment of sequences for comparison are well known in the art. Optimal alignment of sequences for comparison can be conducted, e.g., by the local homology algorithm of Smith and Waterman, Adv. Appl. Math. 2: 482c, 1970; by the homology alignment algorithm of Needleman and Wunsch, J. Mol. Biol. 48:443, 1970; by the search for similarity method of Pearson and Lipman, Proc. Natl. Acad. Sci. USA 85:2444, 1988; by computerized implementations of these algorithms (GAP, BESTFIT, FASTA, and TFASTA in the Wisconsin Genetics Software Package, Genetics Computer Group, Madison, WI); or by manual alignment and visual inspection (see, e.g., Brent et al., Current Protocols in Molecular Biology, John Wiley & Sons, Inc. (ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

The term “subject” refers to human and non-human animals (especially non-human mammals). The term “subject” is used herein, for example, in connection with therapeutic and diagnostic methods, to refer to human or animal subjects. Animal subjects include, but are not limited to, animal models, such as, mammalian models of conditions or disorders associated with elevated ebolavirus expression such as CLL, ALL, mantle cell lymphoma, neuroblastoma, sarcoma, renal cell carcinoma, breast cancer, lung cancer, colon cancer, head and neck cancer, melanoma, and other cancers. Other specific examples of non-human subjects include, e.g., cows, horses, sheep, pigs, cats, dogs, mice, rats, rabbits, guinea pigs, monkeys.

The terms “treat,” “treating,” “treatment,” and “therapeutically effective” used herein do not necessarily imply 100% or complete treatment. Rather, there are varying degrees of treatment recognized by one of ordinary skill in the art as having a potential benefit or therapeutic effect. In this respect, the therapeutic methods described herein can provide any amount of any level of treatment. Furthermore, the treatment provided by the methods can include the treatment of one or more conditions or symptoms of the disease being treated.

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

### III. Fusions Containing a Sialidase and a Bispecific Immune Cell Engager

In one aspect, the invention provides fusion proteins or fusion molecules that contain a sialidase (or enzymatic fragment thereof) that is conjugated or linked to a bispecific immune cell engager molecule. As used herein, a bispecific immune cell engager molecule refers to any bispecific molecules (e.g., bispecific antibodies) that are capable of specifically binding to both (1) a target antigen (e.g., a surface molecule or receptor) on a target cell and (2) an immune cell that can exert an immune activity (e.g., cytotoxicity) against the target cell. Bispecific molecules suitable for the invention can be present in various formats that are well known in the art. See, e.g., Labrijn et al., Nat. Rev. Drug Discovery 18:585-608, 2019. In some embodiments, the immune cell to be engaged by the bispecific molecule is T cell. In some of these embodiments, the employed bispecific immune cell engager is a BiTE. In some other embodiments, the immune cell to be targeted by the bispecific molecule is an innate immune cell, e.g., NK cell or macrophages. In some of these embodiments, the employed bispecific immune cell engager is an innate cell engager. In some other embodiments, the bispecific cell engaging molecule can contain two antigen binding arms that are connected via Fc-mediated heterodimerization, knob-into-hole or other formats. Typically, the bispecific molecule binds to the immune cell via a surface marker antigen on the cell. For example, BiTEs suitable for the invention can bind to an antigen in the TCR complex or a protein associated therewith such as CD3. Similarly, bispecific innate cell engagers (e.g., BiKEs) that can be used in the invention can target any specific surface markers on the innate immune cell, e.g., NKp44 or CD16 on NK cells or macrophage. Specific examples of bispecific innate cell engagers such as BiKEs and their constructions have been known in the art. See, e.g., Pinto et al., Trends Immunol. 43:933, 2022.

Some preferred embodiments of the invention are directed to BiTE-sialidase fusions. Examples of such fusions are set forth in, e.g., SEQ ID NOs: 7, 8, 11, 13 and 15. In some of these embodiments, one of the tandem scFvs in the BiTEs recognizes the CD3 subunit of the T cell receptor complex, and the other one binds to an antigen on tumor cells. Many BiTE molecules and their uses in cancer immunotherapies have been reported in the art. See, e.g., Huehls et al., Immunol. Cell Biol. 93:290-296, 2015; Lejeune et al., Front. Immunol., Vol. 11, Article 762, 2020; Ross et al., PLOS One. 12: e0183390, 2017; Vafa et al., Front. Immunol., Vol. 10, Article 446, 2020; Haber et al., Sci. Rep. 11:14397, 2021; Ellerman, Methods 154:102-117, 2019; Lund et al., BMC Cancer 20:1214, 2020; and Einsele et al., Cancer 126:3192-3201, 2020. Any of these known BiTEs and those exemplified herein can be used to construct BiTE-sialidase fusions. The BiTEs can be readily generated in accordance with the description of the invention or standard protocols routinely practiced in the art. As exemplification, each of the scFvs in the BiTEs can be constructed by connecting the heavy and light chains of each Fv with a serine-glycine linker sequence. As exemplified in the BiTE molecules herein, the linker can be generally constructed of two, three or more SGGGG (SEQ ID NO:34) repeats, making the peptide sufficiently long and flexible to allow the heavy and light chains to associate in a normal conformation. A similar GS linker can be used to connect the two scFvs, e.g., SEQ ID NOs: 1, 2, 29-31, and 34-37 as exemplified herein. The length of this linker determines the flexibility of movement between the two scFvs and can be adjusted by including more or fewer repeats to optimize binding to both target cells. The entire BiTE molecule consists of one continuous polypeptide. In some embodiments, the complete BiTE molecule is approximately 55 kDa in size and approximately 11 nm in length.

In addition to BiTEs, the sialidase fusion proteins of the invention can also contain other types of immune cell engaging bispecific molecules. In some embodiments, a bispecific innate cell engager can be fused to the sialidase. As exemplifications, several bispecific molecules engaging NK cells (i.e., BiKEs) via the CD16A surface marker, and respectively bind to CD19 or EFGR on target cells are described herein. Sequences of fusion proteins containing these BiKEs and a sialidase are set forth in SEQ ID NOs: 23-28, respectively. As described herein, these BIKE-sialidase fusion molecules are capable of selectively desialylating the target cells and also exhibit enhanced cytotoxicity.

In some embodiments, the bispecific engaging molecule in the fusion proteins of the invention contains two antibody fragments (e.g., scFv or tandem V_{H}−V_{L} fragments) that connected via two Fc arms respectively linked to the antibody fragments. In some of these fusion proteins, the two antibody fragments are connected via knob and hole mutations respectively introduced into the two Fc arms. The use of “knob mutations” and “hole mutations” in Fc fusion dimerization is well known in the art. See, e.g., Merchant et al., Nat. Biotechnol. 16, 677-681, 1998; Jendeberg et al., J. Immunol. Methods 201, 25-34, 1997; Ridgway et al., Protein Engineering 9:617, 1996; Rouet et al., Nat. Biotechnol. 32 (2): 136, 2014; and Xu et al., mAbs. 7 (1): 231-242, 2015. For example, the knob and hole mutations engineered for the connection can be a T366Y mutation and a Y407T mutation introduced respectively into the C_{H}3 region of the Fc portion of the two antibody fragments.

Other than the immune cell targeting functionality, the bispecific molecule in the fusion proteins of the invention also recognizes a target antigen that is associated with or implicated in a disease or disorder (e.g., cancer). Typically, the target antigen is from a cell that is implicated in or responsible for the development of the disease. Any surface antigen on such a disease causing cell can be targeted with the bispecific molecule in the sialidase fusions. In some preferred embodiments, the target antigen is selectively or primarily expressed on a tumor cell. In some embodiments, the cell surface molecule to be targeted by the fusion proteins of the invention can be a receptor. The receptor may be an extracellular receptor. The receptor may be a cell surface receptor. By way of non-limiting example, the receptor may bind a hormone, a neurotransmitter, a cytokine, a growth factor or a cell recognition molecule. The receptor may be a transmembrane receptor. The receptor may be an enzyme-linked receptor. The receptor may be a G-protein couple receptor (GPCR). The receptor may be a growth factor receptor. The cell surface molecule may be a non-receptor cell surface protein. The target molecule may be a cluster of differentiation proteins. By way of non-limiting example, the cell surface molecule may be selected from CD19, CD20, CD34, CD31, CD117, CD45, CD11b, CD15, CD24, CD114, CD182, CD14, CD11a, CD91, CD16, CD3, CD4, CD25, CD8, CD38, CD22, CD61, CD56, CD30, CD13, CLL1, CD33, CD123, or fragments or homologs thereof.

In addition to targeting the cancer markers noted above, the sialidase fusions of the invention can also target antigens or neoantigens that are presented by MHC I or MHC II molecules on the surface of tumor cells. In some preferred embodiments, these antigens are presented only by tumor cells and never by the normal ones. In some embodiments, the target antigens are tumor-specific antigens (TSAs) and, in general, result from a tumor-specific mutation. In some embodiments, the target antigens are antigens that are presented by tumor cells and normal cells, i.e., tumor-associated antigens (TAAs). In some embodiments, the target molecule on the tumor cell surface can be a molecule that does not comprise a peptide. The cell surface molecule may comprise a lipid. The cell surface molecule may comprise a lipid moiety or a lipid group. The lipid moiety may comprise a sterol. The lipid moiety may comprise a fatty acid. The antigen may comprise a glycolipid. The cell surface molecule may comprise a carbohydrate.

Bispecific molecules engaging the target cell with the immune cell can be produced by routinely practiced methods. As noted above, BiTEs or bispecific innate cell engagers that target various tumor antigens or other disease associated antigens have been reported in the art. These include various tumor cell surface makers, e.g., Her2, CD19 or PSMA exemplified herein. Bispecific molecules specific for other cancer-targeting bispecific molecules can also be readily produced. Suitable tumor cell surface targets for the bispecific molecules include, e.g., CD33, the EGFR, EGFR vIII, CD66e, EphA2, MCSP (melanoma), the EpCAM antigen (colon, gastric, prostate, ovarian, lung, and pancreatic cancers), CEA, and the gp100 peptide (unresectable or metastatic uveal melanoma).

Any sialidases or enzymatic fragments thereof can be used in the construction of the fusion proteins of the invention. Sialidases (neuraminidases) are glycoside hydrolase enzymes that cleave (cut) the glycosidic linkages of neuraminic acids. These enzymes are a large family, found in a range of organisms. The best-known neuraminidase is the viral neuraminidase, a drug target for the prevention of the spread of influenza infection. The viral neuraminidases are frequently used as antigenic determinants found on the surface of the influenza virus. Some variants of the influenza neuraminidase confer more virulence to the virus than others. Other homologues are found in mammalian cells, which have a range of functions. As described below, at least seven mammalian sialidase homologues and isoforms have been described in the human genome.

In some embodiments, the fusion proteins of the invention contain a human sialidase. A number of human sialidases are known in the art. These include human sialidases NEU1, NEU2, NEU3, NEU4, as well as several isoforms. Sequences of these human enzymes (e.g., SEQ ID NOs: 16-22 herein), their functional characterization, and recombination production have been reported in the literature. See, e.g., Chavas et al., J. Biol. Chem. 280:469-75, 2005; Lipničanová et al., Intl. J. Biol. Macromol. 148:857-868, 2020; Richards et al., Bioorg. Med. Chem., 26:5349-58, 2018; and US Patent Application 2020/0239512. In addition to wildtype human sialidases, variants or mutants of human sialidases, including conservatively modified variants, can also be used in the fusion proteins of the invention. These variants typically have enhanced or substantially the same enzymatic activities as that of the wildtype sialidases. Additionally or alternatively, they can possess other improved properties, e.g., biological or pharmaceutical properties. In some embodiments, the employed sialidase variants are recombinantly produced human sialidase mutants that contain one or more amino acid substitutions a described in WO 2021/003463.

Other than human sialidases, sialidases obtained from other species and modified variants thereof may also be employed in constructing the fusion molecules of the invention. These include, e.g., viral neuraminidases and bacterial neuraminidases. Widely studied viral sialidases are those from influenza viruses (Orthomyxoviridae). Influenza sialidases have been extensively studied, and are functionally and structurally characterized. Other examples of viral sialidases include, e.g., sialidases from the Paramyxoviridae family. See, e.g., Durrant et al., J. Phys. Chem. B. 120:8590-99, 2016; Vavricka et al., Nat. Commun. 4: e1491, 2013; Stelfox et al., Proc. Natl. Acad. Sci. USA 116:21514-20, 2019; and Villar et al., Glycoconj. J. 23:5-17, 2006. Examples of known bacterial sialidases include, e.g., S. typhimurium sialidase, V. cholerae sialidase, B. infantis sialidase, and B. bifidum sialidase. See, e.g., Varghese et al., Proteins: Struct. Funct. Genet. 14:327-32, 1992; Fougerat et al., Mol. Metab., 12:76-88, 2018; Park et al., Biochim. Biophys. Acta, 1834:1510-1519, 2013; Nishiyama et al., mBio 8: e00928-17, 2017; Kaisar et al., mSphere 6: e01232-20, 2021; Prevato et al., PLOS One, 10 (2015), Article e0135474; Cirillo et al., J. Biol. Chem., 291:10615-10624, 2016; and Crennell et al., Structure 2:535-544, 1994.

The method for generating the fusion proteins of the invention is not subject to any particular limitation. The fusion protein of the invention may be a fusion protein synthesized by chemical synthesis, or a recombinant fusion protein produced by a genetic engineering technique. If the fusion protein of the invention is to be chemically synthesized, synthesis may be carried out by, for example, the Fmoc (fluorenylmethyloxycarbonyl) process or the tBoc (t-butyloxycarbonyl) process. In addition, peptide synthesizers available from, for example, Advanced ChemTech, PerkinElmer, Pharmacia, Protein Technology Instrument, Syntheceh-Vega, PerSeptive and Shimadzu Corporation may be used for chemical synthesis. If the fusion protein of the invention is to be produced by a genetic engineering technique, production may be carried out using the conventional recombination techniques routinely practiced in the art. Such techniques are described, e.g., in Sambrook et al., Molecular Cloning: A Laboratory Manual, Cold Spring Harbor Press, N.Y., (3^{rd} ed., 2000); and Brent et al., Current Protocols in Molecular Biology, John Wiley & Sons, Inc. (ringbou ed., 2003). As detailed below, the fusion protein can be produced by inserting a polynucleotide (e.g., DNA) encoding the fusion protein into a suitable expression system.

In some preferred embodiments, the sialidase fusion proteins of the invention are generated in accordance with the routinely practiced recombination technology. Some specific exemplifications are discussed in detail in the Examples below. As exemplified herein, the sialidase can be operably fused at either the N- or C-terminus of the bispecific molecule. Typically, the methods involve removing the stop codon from a polynucleotide sequence (e.g., a cDNA sequence) coding for one of the two fusion components (e.g., BiTE), then appending a polynucleotide sequence (e.g., a cDNA sequence) encoding the other component (e.g., the sialidase) in frame through ligation or overlap extension PCR. To ensure proper folding and maintain the biological activities of the fusion partners, a linker or spacer peptides may be used for linking the two components of the fusion proteins, e.g., a GS rich linker as shown in some of the sialidase fusions described in the Examples herein. The fusion proteins of the invention may additionally include a peptide sequence or tag for purification. Peptide sequences for purification that may be used are also known in the art. As exemplified herein, examples of peptide sequences for purification include histidine tag sequences having an amino acid sequence in which at least four, and preferably at least six, continuous histidine residues, and the amino acid sequence of the glutathione-binding domain in glutathione S-transferase.

### IV. Polynucleotides, Vectors and Host Cells for Producing Sialidase Fusions

Other than the sialidase fusion proteins disclosed above, related embodiments of the invention also include polynucleotide sequences that encode such fusions, expression constructs for expressing the fusion proteins, and host cells that harbor the polynucleotides or expression constructs. Polynucleotides or nucleic acids of the invention encompass deoxyribonucleic acid (DNA) or ribonucleic acid (RNA) sequences. They include, without limitation, messenger RNA (mRNA), DNA/RNA hybrids, or synthetic nucleic acids. In some embodiments, polynucleotides of the invention include small nucleolar RNA (sno-RNA), microRNA (miRNA), small interfering RNA (siRNA) or Piwi-interacting RNA (piRNA). The nucleic acids of the invention may be single-stranded, or partially or completely double-stranded (duplex). Duplex nucleic acids may be homoduplex or heteroduplex.

The recombinant constructs or expression vectors of the invention harbor a polynucleotide sequence of the invention that encodes a sialidase fusion polypeptide. The recombinant constructs of the invention may be obtained by ligating (inserting) the polynucleotide (DNA) of the invention into a suitable vector. More specifically, the recombinant vector may be obtained by cleaving purified polynucleotide (DNA) with a suitable restriction enzyme, then inserting the cleaved polynucleotide to a restriction enzyme site or multicloning site on a suitable vector, and ligating the polynucleotide to the vector. The vector for inserting the polynucleotide sequence is not subject to any particular limitation, provided it is capable of replication in an appropriate host. The expression vectors of the invention are not subject to any particular limitation, and may be, for example, bacteriophages, plasmids, cosmids or phagemids. Examples of recombinant bacteriophage or phagemid vectors include that based on a filamentous phage such as M13. Plasmid vectors include those based on plasmids from, e.g., E. coli (e.g., pBR322, pBR325, pUC118 and pUC119), plasmids from Bacillus subtilis (e.g., pUB110 and pTP5), and plasmids from yeasts (e.g., YEp13, YEp24 and YCp50). The expression vectors can also include animal viruses such as retroviruses, vaccinia viruses and insect viruses (e.g., baculoviruses).

In the expression constructs of the invention, the polynucleotide encoding the sialidase fusion is generally ligated downstream from the promoter in a suitable vector in such a way as to be expressible. For example, if the host during transformation is an animal cell, preferred promoters include promoters from SV40, retrovirus promoters, metallothionein promoters, heat shock promoters, cytomegalovirus promoters and the SRa promoter. If the host is a genus Escherichia organism, preferred promoters include the tetracycline promoter, the Trp promoter, the T7 promoter, the lac promoter, the recA promoter, the 2 promoter and the lpp promoter. If the host is a genus Bacillus organism, preferred promoters include the SPO1 promoter, the SPO2 promoter and the penP promoter. If the host is a yeast, preferred promoters include the PHO5 promoter, the PGK promoter, the GAP promoter, the ADHI promoter and the GAL promoter. If the host is an insect cell, preferred promoters include the polyhedrin promoter and the P10 promoter.

In addition to the above, the recombinant vector used in the invention may contain, if desired, an enhancer, a splicing signal, a poly (A) addition signal, a ribosome binding sequence (SD sequence), a selective marker and the like. Examples of selective markers include the tetracycline resistance gene, the carbencillin resistance gene, the dihydrofolate reductase gene, the ampicillin resistance gene and the neomycin resistance gene. The recombinant vector of the invention may additionally include a polynucleotide having a nucleotide sequence encoding an amino acid sequence for enhancing translation and/or a polynucleotide having a nucleotide sequence encoding a peptide sequence for purification. For example, the vectors can employ a translational enhancer element (TEE) sequence (see, e.g., Batten et al., FEBS Lett. 580:2591-7, 2006).

The invention further provides host cells that express the sialidase fusion polypeptides described herein. The host cells are genetically engineered (transduced, transformed or transfected) with the recombinant constructs or expression vectors disclosed herein for production of the fusion protein or examination of its activity. To generate such cells, a recombinant construct which harbors and expresses a sialidase fusion sequence can be introduced into a suitable host. The engineered host cells can be cultured in conventional nutrient media modified as appropriate for activating promoters, selecting transformants or amplifying particular genes such as the fusion gene encoding a sialidase fusion polypeptide. The culture conditions for particular host cells selected for expression, such as temperature, pH and the like, will be readily apparent to the ordinarily skilled artisan. In some embodiments, the sialidase fusion sequence is stably integrated into the chromosome of the host cells. With such host cells, the sialidase sequence and its expression are substantially maintained in successive generations of cells. They are distinguished from host cells which transiently express the fusion polypeptide as detailed herein.

The host cell for production or expression of a construct of the invention, for example, can be a higher eukaryotic cell, such as a mammalian cell, or a lower eukaryotic cell, such as a yeast cell, or the host cell can be a prokaryotic cell, such as a bacterial cell. The selection of an appropriate host is within the scope of those skilled in the art and also exemplified in the Examples herein. Representative examples of appropriate host cells suitable for practicing the present invention include, but need not be limited to, bacterial cells, such as E. coli, Streptomyces, Salmonella typhimurium; fungal cells, such as yeast; insect cells, such as Drosophila S2 and Spodoptera Sf9; animal cells, such as CHO, COS or 293 cells; adenoviruses; plant cells, or any suitable cell already adapted to in vitro propagation or so established de novo. If the expression construct is a phage or a phagemid vector, many suitable bacterial host cells can be used, e.g., the E. coli ER2738 cell line as detailed in the Examples below. Another example of such host cells is E. coli strain BW310 as exemplified in the Examples below. This cell line is available from the Coli Genetic Stock Center at Yale University (New Haven, CT). BW310 cell doesn't have ung gene (i.e., ung-genotype). The ung gene encodes uracil-DNA glycosylase which prevents mutagenesis by eliminating from DNA molecules uracil bases produced by cytosine deamination or misincorporation of dUMP residues. This BW310 cell line has been routinely employed in the art to study expression and DNA mutator activity of AID or its orthologs (see, e.g., Ichikawa et al., J. Immunol. 177:355-361, 2006; and Haché et al., J. Biol. Chem. 280:10920-4, 2005).

The polynucleotides and related vectors of the invention can be readily generated with standard molecular biology techniques or the protocols exemplified herein. For example, general protocols for cloning, transfecting, transient gene expression and obtaining stable transfected cell lines are described in the art, e.g., Sambrook et al., Molecular Cloning: A Laboratory Manual, Cold Spring Harbor Press, N.Y., (3^{rd} ed., 2000); and Brent et al., Current Protocols in Molecular Biology, John Wiley & Sons, Inc. (ringbou ed., 2003). Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, CA, 1990; Mattila et al., Nucleic Acids Res. 19:967, 1991; and Eckert et al., PCR Methods and Applications 1:17, 1991. More specific teaching of preparing mRNA therapeutics and mRNA vaccines is also provided in the art. For example, detailed guidance for producing therapeutic mRNAs is described in, e.g., U.S. Pat. Nos. 9,464,124; 9,447,164; 9,428,535; 9,334,328; 9,303,079; 9,301,993; 9,295,689; 9,283,287; 9,271,996; 9,255,129; 9,254,311; 9,233,141; 9,221,891; 9,220,792; 9,220,755; 9,216,205; 9,192,651; 9,186,372; 9,181,319; 9,149,506; 9,114,113; 9,107,886; 9,095,552; 9,089,604; 9,061,059; 9,050,297; 8,999,380; 8,980,864; 8,822,663; 8,754,062; 8,710,200; 8,680,069 and 8,664,194.

Introduction of the vector or expression construct into the host cell can be effected by a variety of methods with which those skilled in the art will be familiar, including but not limited to, for example, calcium phosphate transfection, DEAE-Dextran mediated transfection, or electroporation (see, e.g., Brent et al., supra). Expression and, if desired, purification, of a sialidase fusion polypeptide in a transfected or transformed host cell can be carried out in accordance with any of the routinely practiced methods in the art, e.g., Sambrook et al., supra; and Brent et al., supra). Typically, to produce the fusion protein of the invention, the host cell harboring the expression vector is cultured under appropriate conditions that allow the polynucleotide (DNA) encoding the fusion protein to be expressed, thereby inducing formation and accumulation of the fusion polypeptide, then isolating and purifying the fusion polypeptide. The fusion protein expressed in the host cell can be readily isolated and purified. Specifically, when the fusion protein of the invention accumulated within cultured bacteria or within cultured cells, following the completion of cultivation, an extract of the fusion protein of the invention may be obtained by a conventional method such as centrifugation or filtration after using a conventional technique (e.g., ultrasound, lysozymes, freezing and thawing) to disrupt the bacteria or cells. When the sialidase fusion polypeptide accumulates in the periplasmic space, following the completion of cultivation, an extract containing the target protein may be obtained by a conventional method such as osmotic shock. When the fusion protein of the invention accumulates in the culture broth, following the completion of cultivation, a culture supernatant containing the inventive fusion protein may be obtained by using a conventional method such as centrifugation or filtration to separate the culture supernatant from the bacteria or cells.

### V. Therapeutic Applications

The sialidase fusion molecules and related compositions described herein can be used for treating or ameliorating the symptoms of various tumors expressing an antigen that is recognized by the bispecific molecule in the fusion proteins. The sialidase fusion molecules of the invention can be directly administered under sterile conditions to the subject to be treated. The fusion proteins can be administered alone or as the active ingredient of a pharmaceutical composition. Therapeutic composition of the present invention can be combined with or used in association with other therapeutic agents. Various cancers can be treated with the methods of the invention. These include cancers derived from any tissue such as, e.g., a tissue of a brain, an esophagus, a breast, a colon, a lung, a glia, an ovary, a uterus, a testicle, a prostate, a gastrointestinal tract, a bladder, a liver, a thyroid and skin. In some embodiments, the cancer to be treated is derived from bone. In some embodiments, the cancer to be treated is derived from blood. In these embodiments, the cancer can be derived from a B cell, a T cell, a monocyte, a thrombocyte, a leukocyte, a neutrophil, an eosinophil, a basophil, a lymphocyte, a hematopoietic stem cell or an endothelial cell progenitor. In some embodiments, the cancer can be derived from a CD19-positive B lymphocyte. In some embodiments, the cancer may be derived from a stem cell. For example, the targeting cancer cell may be derived from a pluripotent cell. In some embodiments, the cancer cell to be targeted can be derived from one or more endocrine glands. The endocrine gland may be a lymph gland, pituitary gland, thyroid gland, parathyroid gland, pancreas, gonad or pineal gland.

Many tumors or cell proliferative disorders can be treated with methods of the invention. These include solid tumors, lymphomas, leukemias and liposarcomas. The disorders to conditions to be treated can be acute, chronic, recurrent, refractory, accelerated, in remission, stage I, stage II, stage III, stage IV, juvenile or adult. Solid tumors that can be treated with methods of the invention include, e.g., cancers originated or derived from a brain, an esophagus, a breast, a colon, a lung, a glia, an ovary, a uterus, a testicle, a prostate, a gastrointestinal tract, a bladder, a liver, a thyroid and skin.

In some embodiments, the cancer to be treated is heterogeneous. In some embodiments, the cancer to be treated is a blood cell malignancy. For example, the cancer to be treated can be derived from bone marrow cells or other blood cells. In these embodiments, the cancer can be derived from a B cell, a T cell, a monocyte, a thrombocyte, a leukocyte, a neutrophil, an eosinophil, a basophil, a lymphocyte, a hematopoietic stem cell or an endothelial cell progenitor. In some embodiments, the cancer can be derived from a CD19-positive B lymphocyte. In some embodiments, the cancer may be derived from a stem cell. For example, the targeting cancer cell may be derived from a pluripotent cell. In some embodiments, the cancer cell to be targeted can be derived from one or more endocrine glands. The endocrine gland may be a lymph gland, pituitary gland, thyroid gland, parathyroid gland, pancreas, gonad or pineal gland.

In some embodiments, the cancer to be treated is a Her2-positive cancer. These include, e.g., Her2-positive breast cancer and Her2-positive pancreatic cancer. In some embodiments, the cancer to be treated can be a PSMA-positive prostate cancer. In some embodiments, the cancer to be treated is a CD19-positive tumor or malignancy. In some of these embodiments, the cancer to be treated is a B cell cancer or B cell malignancy. B cell cancer or B cell malignancy encompass B-cell lymphomas which account for a major portion of non-Hodgkin lymphomas (NHL). Examples of these B cell cancers include, e.g., diffuse large B-cell lymphoma (DLBCL), follicular lymphoma, chronic lymphocytic leukemia (CLL), small lymphocytic lymphoma (SLL), mantle cell lymphoma (MCL), marginal zone lymphoma, Burkitt lymphoma, lymphoplasmacytic lymphoma (Waldenstrom macroglobulinemia), hairy cell leukemia (HCL), primary central nervous system (CNS) lymphoma, and primary intraocular lymphoma.

The sialidase fusions described herein can be used in combination with other known regimens for treating cancers. These include known antitumor drugs (antineoplastic drugs), tumor metastasis-inhibitors, inhibitors for thrombogenesis, therapeutic drugs for joint destruction, analgesics, anti-inflammatory drugs, immunoregulators (or immunomodulators) and/or immunosuppressants, which can be employed as not being restricted to particular species as long as they serve effectively or advantageously. Methods for co-administration with an additional therapeutic agent are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.).

### VI. Pharmaceutical Compositions

The invention further provides pharmaceutical compositions that contain a sialidase fusion protein described herein and a pharmaceutically acceptable carrier. Pharmaceutical compositions can be prepared from any of the sialidase fusion molecules described herein. The pharmaceutically acceptable carrier can be any suitable pharmaceutically acceptable carrier. It can be one or more compatible solid or liquid fillers, diluents, other excipients, or encapsulating substances which are suitable for administration into a human or veterinary patient (e.g., a physiologically acceptable carrier or a pharmacologically acceptable carrier). The term “carrier” denotes an organic or inorganic ingredient, natural or synthetic, with which the active ingredient is combined to facilitate the use of the active ingredient, e.g., the administration of the active ingredient to a subject. The pharmaceutically acceptable carrier can be co-mingled with one or more of the active components, e.g., an adapter molecule, and with each other, when more than one pharmaceutically acceptable carrier is present in the composition, in a manner so as not to substantially impair the desired pharmaceutical efficacy. Pharmaceutically acceptable materials typically are capable of administration to a subject, e.g., a patient, without the production of significant undesirable physiological effects such as nausea, dizziness, rash, or gastric upset. It is, for example, desirable for a composition comprising a pharmaceutically acceptable carrier not to be immunogenic when administered to a human patient for therapeutic purposes.

Pharmaceutical compositions of the invention can additionally contain suitable buffering agents, including, for example, acetic acid in a salt, citric acid in a salt, boric acid in a salt, and phosphoric acid in a salt. The compositions can also optionally contain suitable preservatives, such as benzalkonium chloride, chlorobutanol, parabens, and thimerosal. Pharmaceutical compositions of the invention can be presented in unit dosage form and can be prepared by any suitable method, many of which are well known in the art of pharmacy. Such methods include the step of bringing the antibody of the invention into association with a carrier that constitutes one or more accessory ingredients. In general, the composition is prepared by uniformly and intimately bringing the active agent into association with a liquid carrier, a finely divided solid carrier, or both, and then, if necessary, shaping the product.

A composition suitable for parenteral administration conveniently comprises a sterile aqueous preparation of the inventive composition, which preferably is isotonic with the blood of the recipient. This aqueous preparation can be formulated according to known methods using suitable dispersing or wetting agents and suspending agents. The sterile injectable preparation also can be a sterile injectable solution or suspension in a non-toxic parenterally-acceptable diluent or solvent, for example, as a solution in 1,3-butane diol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed, such as synthetic mono- or di-glycerides. In addition, fatty acids such as oleic acid can be used in the preparation of injectables. Carrier formulations suitable for oral, subcutaneous, intravenous, intramuscular, etc. administrations can be found in Remington's Pharmaceutical Sciences, Mack Publishing Co., Easton, PA.

Preparation of pharmaceutical compositions of the invention and their various routes of administration can be carried out in accordance with methods well known in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. The delivery systems useful in the context of the invention include time-released, delayed release, and sustained release delivery systems such that the delivery of the inventive composition occurs prior to, and with sufficient time to cause, sensitization of the site to be treated. The inventive composition can be used in conjunction with other therapeutic agents or therapies. Such systems can avoid repeated administrations of the inventive composition, thereby increasing convenience to the subject and the physician, and may be particularly suitable for certain compositions of the invention.

Many types of release delivery systems are available and known to those of ordinary skill in the art. Suitable release delivery systems include polymer base systems such as poly (lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and triglycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

Some embodiments of the invention are directed to pharmaceutical compositions or delivery formulations that contain mRNA molecules or mRNA sequences that encode a sialidase fusion protein described herein. The mRNA sequences can be directly employed in therapeutic applications as efficacious mRNA vaccines. The mRNA molecules can be optionally formulated with additional components designed to facilitate efficacious delivery of mRNAs in vivo, e.g., complexed with polymeric or lipid components. In some embodiments, the pharmaceutical compositions of the invention can contain (i) an effective amount of a synthetic mRNA encoding a sialidase fusion protein described herein; (ii) a cell penetration agent; and (iii) a pharmaceutically acceptable carrier. In these embodiments, the mRNA may contain pseudouridine, 5′methyl-cytidine or a combination thereof. In some of these embodiments, the mRNA does not contain a substantial amount of a nucleotide or nucleotides selected from the group consisting of uridine, cytidine, and a combination of uridine and cytidine.

As exemplified herein, some embodiments of the invention are directed to pharmaceutical compositions that contain a mRNA molecule of the present invention formulated as a lipid nanoparticle (LNP) formulation, e.g., with a PEG lipid, PEG lipids have been used in many pharmaceutical compositions, cosmetic compositions, and drug delivery systems. Other than the lipid materials exemplified herein, the LNPs described in US Patent Publication Nos. 20220047518 and 20200254086 can also be adapted and modified for the delivery of an mRNA agent of the invention to a subject. In some embodiments, the lipid nanoparticle formulation of the invention contains lipids including an ionizable lipid (such as an ionizable cationic lipid), a structural lipid, a phospholipid, and the mRNA agent. In some embodiments, the lipid nanoparticle contains an ionizable lipid, a PEG-modified lipid, a phospholipid and a structural lipid.

In some other embodiments, chemical modifications can be introduced into the mRNA sequences to promote certain desirable properties of the vaccines, e.g., to reduce unwanted innate immune responses against mRNA components and/or to facilitate desirable levels of protein expression. In various embodiments, chemical modifications of the mRNA sequences include the use of one or more chemically modified ribonucleosides or analogs. In some embodiments, the present invention provides a packaged pharmaceutical composition for treating tumors such as a kit or other container. Typically, the kit or container holds a therapeutically effective amount of a sialidase fusion protein or polynucleotide described herein. The kit can optionally contain an instruction sheet detailing how to use the fusion molecule to treat cancer.

### EXAMPLES

The following examples are provided to further illustrate the invention but not to limit its scope. Other variants of the invention will be readily apparent to one of ordinary skill in the art and are encompassed by the appended claims.

### Example 1. Tumor Cell-Surface Sialic Acids Removal Enhances BiTE-Mediated Tumor Cell Killing by T Cells

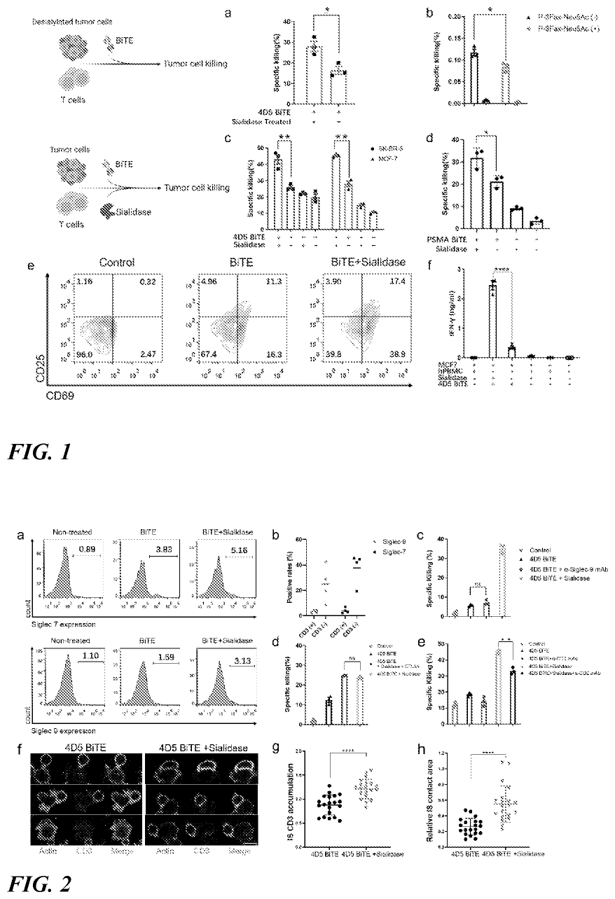

To evaluate whether desialylation may enhance the susceptibility of tumor cells to BiTE-mediated cytotoxicity by T cells, we first constructed a BiTE molecule from a HER2-targeting scFv 4D5 and a human CD3-targeting scFv (4D5 BiTE). We then treated HER2 positive SK-BR-3 human breast cancer cells with a sialidase derived from Bifidobacterium longum subspecies infantis (B. infantis) to remove cell surface sialic acids. Following this procedure, treated cells were incubated with PBMCs from healthy human donors in the presence of 4D5 BiTE. Staining with FITC-Sambucus nigra agglutinin (SNA) that binds preferentially to sialic acid attached to terminal galactose in an a-2,6 linkage confirmed the success of cell surface desialylation. As shown in FIG. 1, a, desialyation significantly potentiated the 4D5 BiTE-induced tumor cell killing by T cells compared with 4D5 BiTEs alone. A similar trend was observed when 4D5 BiTE treatment was combined with a sialylation inhibitor, P-3Fax-Neu5Ac (FIG. 1, b). Next, we treated SK-BR-3 and MCF-7 breast cancer cells with 4D5 BiTE and hPBMCs in the presence or absence of B. infantis sialidase, respectively. Significantly, the addition of sialidase triggered stronger killing of both cell lines as compared to both the 4D5 BiTEs+hPBMCs and sialidase+hPBMCs controls (FIG. 1, c). The cytotoxicity enhancement is also observed with a different E: T ratio of 1:1 and the improvement of the BiTE-induced killing was sialidase dose-dependent. To verify the benefits of sialidase treatment for BiTE-induced killing, another BiTE molecule (PSMA BiTE) targeting Prostate-Specific Membrane Antigen (PSMA) was constructed and stronger cytotoxicity enhancement were observed when sialidase and BiTEs were added at the same time (FIG. 1, d). Since BiTEs only engage T cells to achieve tumor cell killing, we repeated the killing assay with MCF-7 cells using purified T cells and confirmed that the sialidase treatment indeed led to better BiTE-mediated target cell killing. Moreover, addition of sialidase also significantly enhanced T cell activation and IFN-γ secretion (FIG. 1, e and f).

### Example 2. Tumor Desialylation Promotes Stronger Immune Synapse (IS) Formation Between T Cells and Tumor Cells

To elucidate the mechanism underlying the potentiation of BiTE-induced cytotoxicity by desialylation, we first investigated if the sialoglycan-Siglec (Sialic acid-binding immunoglobulin-type lectins) inhibitory pathway is involved. Through their interaction with sialylated glycans aberrantly expressed on tumor cells, immune cell-associated Siglecs trigger signaling cascades to suppress immune cell activation and effector functions. Consistent with previous reports, T cells from PMBCs of healthy donors expressed negligible levels of Siglec-7 and Siglec-9 as compared to their CD3 negative counterparts that mainly consist of B cells, NK cells, monocytes and dendritic cells (FIG. 2, b). Nevertheless, we did observe a slight up-regulation of both Siglec-9 and Siglec-7 following T cell activation. When compared to the expression of these Siglecs on freshly isolated CD3 negative cells, the expression of Siglec-7 and -9 on activated T cells was still minimal. To further test if the Siglec-9 inhibitory pathway plays a role in BiTE-induced T cell killing, a Siglec-9 blocking antibody was added with 4D5 BiTEs, and the level of target cell killing was analyzed. In contrast to sialidase addition, blocking of the Siglec-9 signals did not increase cytotoxicity, indicating a negligible role of Siglec-9 in the BiTE-induced T cell killing (FIG. 2, c). To investigate whether the enhanced BiTE-induced killing seen with desialylation is affected by CD28 co-stimulation, a high affinity ligand of CD80, recombinant human CTLA-4, was added to determine if the enhanced cytolysis by desialylation would be attenuated. However, even with the addition of high concentrations of CTLA-4 no change in the enhanced cytolysis was detected (FIG. 2, d).

Formation of the BiTE-induced immunological synapse (IS) between target cells and T cells is the essential mode of action of BiTEs. We hypothesized that the removal of cell-surface sialosides may promote stronger BiTE-induced IS formation between target tumor cells and T cells and thus better tumor cell killing. Accumulation of the TCR-CD3 complex and F-actin at the synapse is a hallmark of a stable and functional cytolytic IS in T cells. To test whether desialylation can promote IS formation, we imaged the IS formed between T cells and sialidase-treated and non-treated SK-BR-3 cells by staining F-actin and CD3. The resulting immunofluorescence was imaged by confocal microscopy. As shown in FIG. 2f, we observed larger BiTE-induced IS formation between T cells and desialylated SK-BR-3 cells. To assess the stability of the IS formed, we calculated the relative CD3 fluorescence intensity at the IS and the relative area of the IS. The IS formed by sialidase-treated tumor cells and T cells showed significantly stronger CD3 accumulation and larger IS area compared to IS formed by untreated tumor cells and T cells (FIG. 2, g and h). The same trend was observed for BiTE-induced IS formation between SKOV-3 cells and T cells, with better IS formed following sialidase treatment.

The interaction between CD2 and CD58 is known to play a critical role in the formation of a productive immunological synapse. We found that the inhibition of this interaction with an anti-CD2 blocking antibody partially reversed the cytotoxicity enhancement from the sialidase addition, strongly suggesting that the desialylation triggers stronger target cell killing by facilitating a tighter interaction between target tumor cells and T cells (FIG. 2, e).

### Example 3. HER2-Targeting BiTE-Sialidase Fusion Protein Selectively Desialylates HER2-Positive Cells

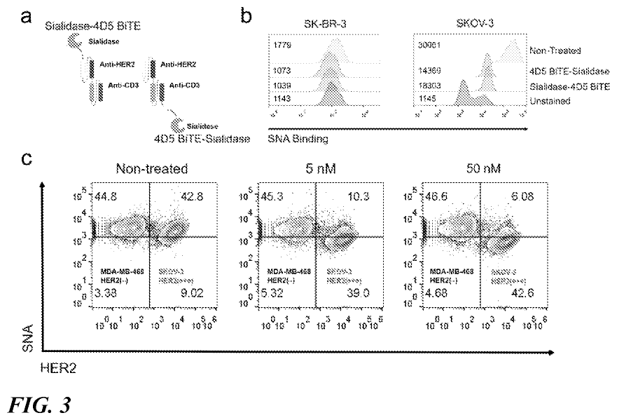

Having confirmed that sialidase treatment potentiates T cell-dependent tumor cell cytolysis induced by BiTE, we next sought to specifically direct sialidase to the tumor cell-T cell interface via BiTE conjugation. Confining sialidase activity to the target cells would potentiate tumor cell killing while limiting nonspecific desialylation of cells in the immune system. Importantly, sialyl-Lewis X, a sialylated tetrasaccharide, is essential for leukocyte tethering and rolling en route to sites of inflammation and tumor tissues. Nonspecific desialylation would destroy this glycan epitope on leukocytes, thereby hindering their tumor homing, and accordingly effective tumor control. Toward this end, we constructed 4D5 BITE-B. infantis sialidase fusion proteins in which sialidase was introduced onto either the N terminus (sialidase-4D5 BiTE) or the C terminus (4D5 BiTE-sialidase) of 4D5 BiTE, respectively (FIG. 3, a). To test whether the fusion protein can successfully remove sialic acids from the surface of tumor cells, SK-BR-3 (HER2+++) and SKOV-3, a human ovarian adenocarcinoma cell line (HER2+++), were treated with Sialidase-4D5 BiTE or 4D5 BiTE-sialidase, respectively, followed by staining with the a-2,6-sialic acid-binding lectin SNA. Desialylation was evidenced by the decreased SNA binding as compared with the untreated controls and was seen in both SK-BR-3 and SKOV-3 cells when treated with either fusion protein (FIG. 3, b). To determine whether the sialidase fusion proteins can selectively desialylate HER2 positive cells in the presence of HER2 negative cells, we mixed SKOV-3 (HER2+++) and MDA-MB-468 (HER2-) cells, followed by the addition of 4D5 BiTE-sialidase. We observed that 4D5 BiTE-sialidase, at both 5 nM and 50 nM concentrations, selectively desialylates HER2 positive SKOV-3 cells while sparing the HER2 negative MDA-MB-468 cells, thus, confirming its selectivity for HER2-expressing cells (FIG. 3, c).

### Example 4. Anti-HER2 BiTE-Sialidase Triggers Enhanced T Cell-Dependent In Vitro Cytotoxicity and T Cell Effector Function than HER2 BITE

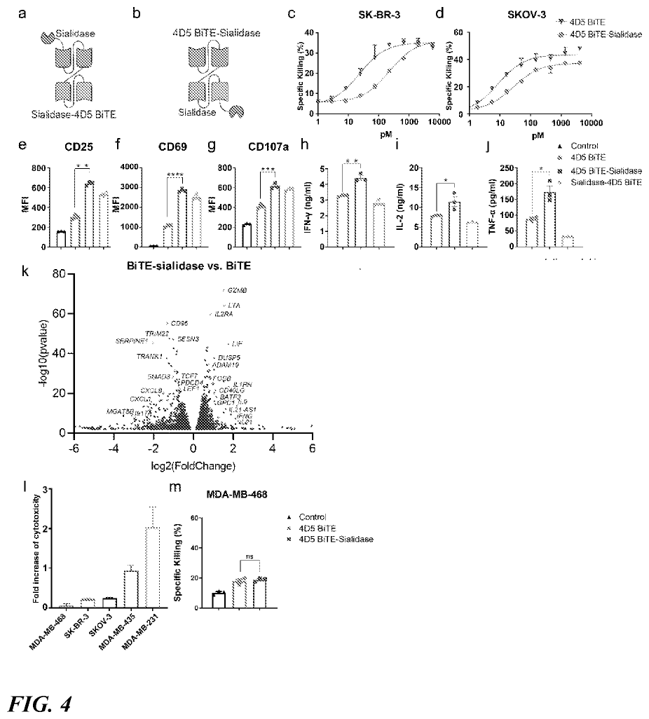

We then compared the T cell-dependent cytotoxicity mediated by both fusion proteins to that of the original 4D5 BiTE. At the same concentration of 4 nM, both fusion proteins induced a higher level of T cell-dependent cytolysis of SK-BR-3 and SKOV-3 cells than 4D5 BiTE (FIG. 4, a and b). Specifically, in a dose-response assay, using SK-BR-3 and SKOV-3 cells as the target cells a tenfold and a threefold lower EC50, respectively, were measured for 4D5 BiTE-sialidase than 4D5 BiTE (4D5 BITE EC50=˜200 pM) (FIG. 4, c and d). Consistent with these findings, 4D5 BiTE-sialidase induced the highest levels of T cell activation as measured by the expression of T cell activation markers CD25 and CD69 and the degranulation marker CD107a (FIG. 4, e to g). Also, the strongest release of cytokines, including IL-2, IFN-γ and TNF-x, was observed for 4D5 BiTE-sialidase-treated T cells. By contrast, sialidase-4D5 BiTE unexpectedly reduced the production of pro-inflammatory cytokines by T cells. (FIG. 4, h to j). A similar trend was seen for SKOV-3 cells, with 4D5 BiTE-sialidase inducing the strongest T cell activation. Consequently, 4D5 BiTE-sialidase was chosen for further studies.

The above studies showed that the 4D5 BiTE-sialidase engaged T cells are better activated versus those engaged by 4D5 BiTE. Therefore, it was of interest to determine whether the better T cell activation was originated from transcriptional alterations induced by BiTE treatment. To systematically characterize transcriptional changes in BiTE-molecule engaged T cells, we performed whole transcriptome RNA-sequencing (RNA-seq) analysis on either the 4D5 BiTE-sialidase or the 4D5 BiTE treated CD3+ T cells co-cultured with target MDA-MB-231 cells. Volcano plot messenger RNA (mRNA) comparisons between the 4D5 BiTE-sialidase and the 4D5 BiTE treated T showed that 1191 transcripts were differentially expressed between these two groups (p<0.01, log 2 (fold change)>0.5) (FIG. 4, k). The most highly expressed gene transcript in 4D5 BiTE-sialidase treated T cells encoded molecules crucial for T cell effector functions, including cytolytic enzymes and cytokines (GZMB, LTA, LIF, IFNG), cytokine receptors (IL2RA), and transcriptional regulators (FOSB, BATF3). Notably, gene transcripts associated with memory phenotypes, such as LEFI and TCF7, inhibitory receptors, such as CD96 and PDCD4, and molecules involved in regulatory T cell generation, e.g., SMAD3, were largely downregulated. Gene-set-enrichment analysis (GSEA) highlighted multiple key pathways that are upregulated in the 4D5 BiTE-sialidase treated T cells, including those associated with cell cycle, transcriptional activity, and cell metabolism. Significantly, expression of transcripts involved in both oxidative phosphorylation and glycolysis was notably increased. By contrast, enrichment of downregulated genes pertained to pathways associated with Wnt-β catenin and TGF-β signaling. Consistently, cytokine signaling (Cytosig) analysis revealed that pro-proliferation and inflammatory cytokines, IL-2, IL-12, IL-15, had the most clearly increased activity in the 4D5 BiTE-sialidase treated T cells, whereas the activity of the suppressive cytokine TGF-B3 was downregulated. Together, compared with the T cells that were treated with 4D5 BiTE, the 4D5 BiTE-sialidase-treated T cells were in a more effector-differentiated state with higher oxidative phosphorylation, glycolysis activities and effector functions.

We further tested the BiTE-sialidase-mediated killing of cell lines with different cell-surface HER2 expression levels: MDA-MB-231 (+), MDA-MB-435 (+) and MDA-MB-468 (−). At 4 nM concentration, compared to 4D5 BiTE, 4D5 BiTE-sialidase strongly augmented the killing of cells with low levels of HER2 (HER2+), e.g., MDA-MB-231 and MDA-MB-435. Under this condition, stronger enhancements in killing were achieved than those measured for HER high (HER2+++) cells (SK-BR-3 and SKOV-3 cells) (94-203% vs. 22-24%) (FIG. 4, 1). These observations suggest that desialylation can increase the susceptibility of cells that would normally be relatively resistant to BiTE-mediated T cell killing. Significantly, 4D5 BiTE-sialidase did not trigger the killing of HER2 negative MDA-MB-468 cells or murine melanoma B16-F10 cells that express abundant sialoglycans, indicating exclusive specificity towards HER2 positive cells (FIG. 4, m)

### Example 5. BiTE-Sialidase Fusion Proteins Specific for CD19 and PSMA Trigger Enhanced In Vitro Cytotoxicity and T Cell Activation

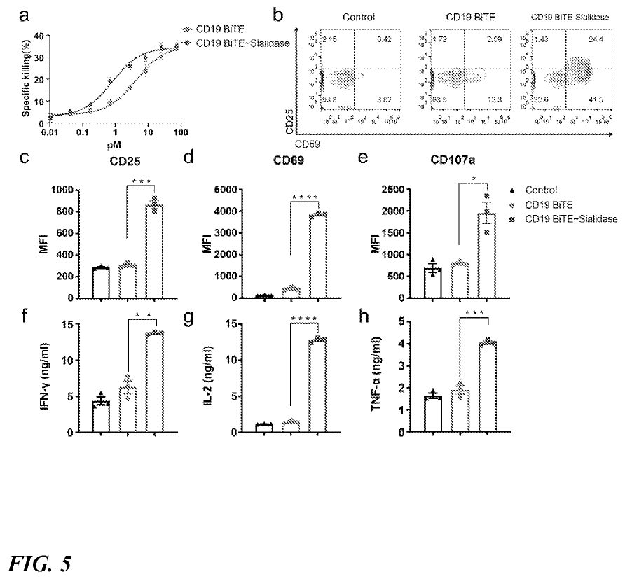

To evaluate whether BiTE-sialidase fusion format can be applied to improve the efficacy of BiTE molecules targeting other tumor-associated antigens, we designed and constructed two additional BiTE-sialidase molecules. The first was based on the FDA-approved drug Blinatumomab that targets CD19, a cell surface marker on B cells and B cell malignancies. The second was derived from BiTE against prostate-specific membrane antigen (PSMA), a target for prostate cancer treatment. As shown in FIG. 5, a, compared to Blinatumomab (CD19 BiTE), the sialidase fusion counterpart exhibited much stronger cytotoxicity toward CD19-positive Raji cells with a fivefold lower EC50 (0.80 pM vs. 4.26 pM). Using the same concentration of 5 pM, CD19 BiTE-sialidase induced much higher T cell activation and degranulation than Blinatumomab (FIG. 5, b to e). Consistent with better T cell activation, CD19 BiTE-sialidase also triggered stronger cytokine release (FIG. 5, f to h). Likewise, much stronger killing of NALM-6, another CD19-positive cell line, was achieved by CD19 BiTE-sialidase. As what we observed for CD19 BiTE-sialidase, PSMA BiTE-sialidase also induced better killing of PSMA-positive PC3 cells and stronger T cell activation in comparison to PSMA BITE.

### Example 6. BiTE-Sialidase Enables Better Tumor Control than BiTE in Xenograft Models in Immune Deficient Mice

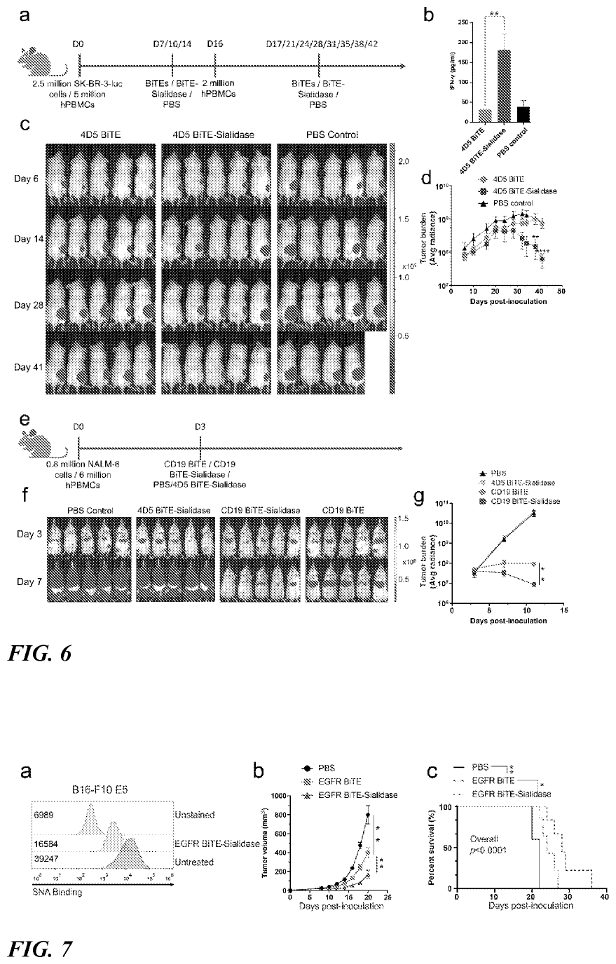

Having demonstrated the superiority of BiTE-sialidase fusion proteins versus the original BiTE molecules in terms of inducing T cell-dependent cytolysis of tumor cells in vitro, we then sought to determine if this enhanced efficacy could also be achieved in vivo. We chose a human tumor murine xenograft model using the NOD-Prkdc^{em26Cd52}IL.2rg^{em26Cd22}/NjuCrl coisogenic (NCG) immunodeficient mouse to compare the antitumor immunity induced by 4D5 BiTE-sialidase and 4D5 BiTE constructs (NCG; CRL572; Charles River Laboratories). On day 0, NCG mice were injected subcutaneously (s.c.) with 2.5 million SK-BR-3-luc cells followed by intraperitoneal (i.p.) administration of 5 million hPBMCs. On day 7, these NCG mice were divided into three groups and then received an intravenous (i.v.) infusion of PBS, 4D5 BiTE, or 4D5 BiTE-sialidase, respectively (FIG. 6, a). After 5 hrs, blood was collected from each mouse to measure the serum IFN-γ level. The 4D5 BiTE-sialidase group was found to harbor the highest level of serum IFN-γ, whereas the 4D5 BiTE-treated group barely had any increased IFN-γ levels over the PBS control group (FIG. 6, b). The BiTE administration was continued twice per week until day 41. A second dose of 2 million hPBMCs per mouse was given on day 16. During this treatment course, tumor growth was monitored by longitudinal, noninvasive bioluminescence imaging. As shown in FIGS. 6, c and d, the administration of 4D5 BiTE-sialidase significantly delayed tumor cell growth in vivo compared to the 4D5 BiTE treatment and PBS control. Remarkably, by the end of the treatment regimen, tumors in two mice receiving the 4D5 BiTE-sialidase treatment were completely eradicated. We next investigated the in vivo efficacy of the CD19 BiTE-sialidase fusion protein using an orthotopic xenograft mouse model of leukemia. In this model, CD19+NALM-6 cells (0.8 million) and hPBMCs (6 million) were injected intravenously (i.v.) into NCG mice on Day 0 (FIG. 6, e). The recipient mice were divided into four groups on day 3 and received i.v. infusion of PBS, 4D5 BiTE-sialidase, CD19 BiTE, or CD19 BiTE-sialidase, respectively. Significantly slower tumor progression was observed in the CD19 BiTE-sialidase treated group as compared with the CD19 BiTE treated group, demonstrating better in vivo antitumor effects of the sialidase fusion protein (FIG. 6, f and g). Notably, no apparent differences were detected between the PBS control group and the group that received non-CD19 targeting 4D5 BiTE-sialidase, indicating that the fusion protein triggered antitumor effects rely on the target engagement on tumor cells (FIG. 6, g).

### Example 7. In Vivo Activities of BiTE-Sialidase Fusion in Melanoma Animal Model

We further observed therapeutic advantages of a BiTE-sialidase fusion protein over the parent BiTE in a syngeneic mouse model of melanoma. Specifically, to evaluate the efficacy of BiTE-sialidase fusion proteins in an immune-competent syngeneic mouse model, we constructed a murine CD3-engaging BiTE and the corresponding BiTE-sialidase from the ScFv fragments derived from anti-human EGFR antibody Cetuximab and anti-murine CD3& clone 17A2. A mouse melanoma cell line, B16-EGFR5 (B16-E5), with the expression of a chimeric mouse EGFR with six amino acid mutations to enable the binding of Cetuximab was chosen as the target cell. The fusion protein successfully induced desialylation of B16-E5 cells in vitro as confirmed by SNA staining (FIG. 7, a). To compare the anti-tumor activities of EGFR BiTE and EGFR BiTE-sialidase in vivo, we inoculated C57BL/6J mice with B16-E5 tumor cells (s.c) followed by intra-tumoral administration of EGFR BiTE or EGFR BITE-sialidase. While both groups conferred therapeutic advantages over the PBS control group, the EGFR BiTE-sialidase treatment significantly delayed tumor growth as compared to the EGFR BiTE counterpart, in addition to offering notable survival benefits to the recipient mice (FIG. 7, b and c). To profile the key effector cell type mediating the tumor control of EGFR BiTE-sialidase treatment, we depleted the CD8+ or CD4+ T-cell population in mice inoculated with B16-E5 tumor cells and treated different groups with the fusion protein (FIG. 14, a). The results indicate that depletion of CD8+ T cells completely abolished the anti-tumor benefit of the EGFR BITE-sialidase treatment, whereas depletion of CD4+ T cells had minimal effects (FIG. 14, b).

Next, we investigated whether BiTE sialidase fusion protein conferred better tumor control by inducing changes in immune cell compositions in the tumor microenvironment. A single high dosage of EGFR BiTE or EGFR BiTE-sialidase was injected intratumorally on Day 11 post-tumor inoculation. Tumors and tumor-draining lymph nodes were harvested three days after the treatment, at which point, the fusion protein-treated group had smaller tumor sizes compared to the BiTE treated group. We found that in tumor-draining lymph nodes of both the EGFR BiTE and the EGFR BiTE-sialidase treated groups had significantly higher numbers of lymphocytes as compared with the PBS control group with the BiTE-sialidase treated group having the highest CD8⁺ T cell counts. When analyzing tumor-infiltrating immune cells, compared with the EGFR BiTE treated groups, the BiTE-sialidase treated group had significantly higher frequencies of CD8⁺ T cells and NK cells (CD45.2⁺CD3⁻NK1.1⁺) and a reduced frequency of myeloid cells (CD45.2⁺CD11b⁺ NK1.1⁻). However, no apparent differences in CD4⁺ T cells and dendritic cells (CD45.2⁺CD11c⁺) were observed.

We then analyzed CD8⁺ T cells in different groups and found that CD8⁺ T cells in the EGFR BiTE-sialidase treated group are skewed to a more effector-like phenotype. Together, these results demonstrated that the BiTE-sialidase fusion protein facilitates the conversion of a myeloid-rich, T cell-poor tumor microenvironment that is immunosuppressive into a more immunopermissive one populated with NK and CD8⁺ T cells, which, in turn, leads to significantly improved tumor control.

### Example 8. Desialylation Efficiency of BiTE-Sialidase Fusions Vs Free Sialidase

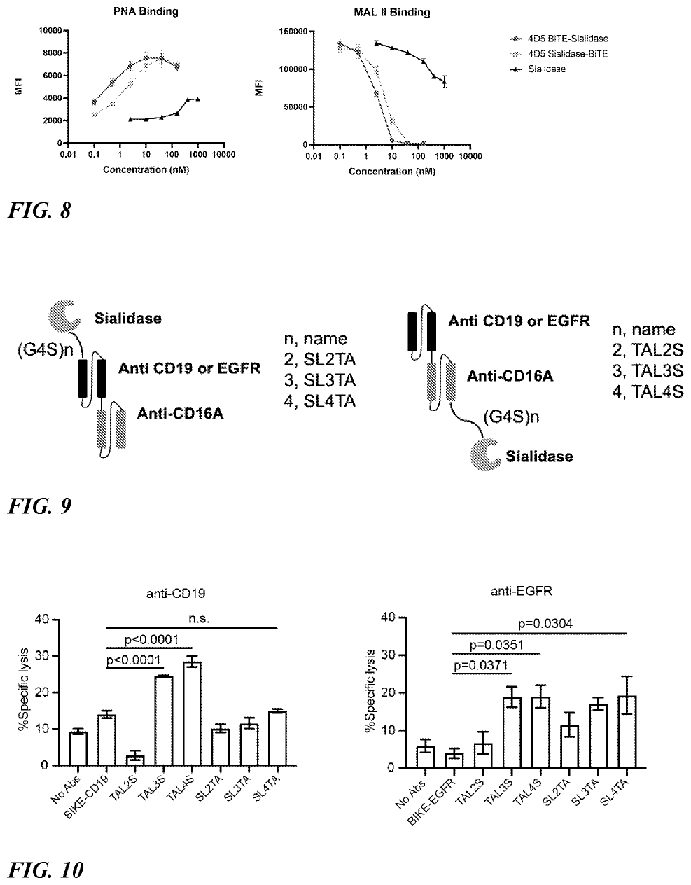

We further compared desialylation efficiency of both our 4D5 BiTE and sialidase fusion proteins and the free sialidase. We measured the binding of two lectins PNA, detecting unsialylated galactose residues, and MAL II, specific for α2-3-linked sialic acid, to the target cells under different concentrations of three proteins. Results of the comparison study are shown in FIG. 8. As can be seen from the figure, both fusion protein constructs showed better desialylation compared to the free sialidase, demonstrating the benefits of targeting the tumor surface for stronger sialic acid removal. In addition, the C terminus conjugated format 4D5 BiTE-sialidase showed better desialylation efficiency than the N terminus conjugated version. This might explain the cytotoxicity and T cell activation discrepancies that have been observed between two constructs.

### Example 9. BiTE Sialidase Fusion Selectively Engaging Cytolytic T Cells

BiTEs that redirect T cells via CD3-binding have demonstrated promising therapeutic potential, as noted above. Nonetheless, they could indiscriminately stimulate both cytolytic T cells and immunosuppressive regulatory T cells (Treg). We therefore also constructed bispecific gammadelta T cells (γδT) cell engager-sialidase fusion proteins that selectively engage Vγ9Vδ2 T cells. Gamma-delta (γδ) T cells are a subset of T cells that promote the inflammatory responses of lymphoid and myeloid lineages, and are especially vital to the initial inflammatory and immune responses. They contain a yδ T-cell receptor (TCR) on their surface as opposed to the αβ TCR on most T cells. For more information about γδT cells in general and Vγ9Vδ2 T cells in particular, see, e.g., Reis et al., Science 377:276-284, 2022; Kabelitz, Cells 9:2564, 2020; and Lin et al., Signal Transduct. Target Ther. 5:215, 2020. Thus, unlike the traditional αβ T cells engaging molecules that work through CD3 binding, γδ T cells engagement is usually achieved by γδ TCR targeting.

An exemplary BiTE-sialidase fusion that engages Vγ9Vδ2 T cells and tumor marker Her2 is shown in SEQ ID NO:41. The BiTE molecule in this fusion protein, 4D5-7A5 (SEQ ID NO:40), was constructed with a y8 TCR targeting scFv, 7A5 (SEQ ID NO:39) and the Her2 targeting scFv 4D5 (SEQ ID NO:3). The BiTE sequence was then fused to B. infantis sialidase (SEQ ID NO:5) with a suitable GS linker, e.g., (GGGGS) 2 (SEQ ID NO: 29) as exemplified herein. Other than the exemplified linker, the γδ T cell engaging BiTE-sialidase fusion proteins of the invention can readily employ other suitable GS linkers described herein, e.g., (GGGGS) 3 (SEQ ID NO:30) or (GGGGS) 4 (SEQ ID NO:31). These bispecific T cell engaging fusion molecules could possess potent anti-tumor properties without suppressive functions. See, e.g., Park et al., Exp Mol Med. 53:318-27, 2021.

### Example 10. In Vitro and In Vivo Activities of BiKE-Sialidase Fusions

Other than BiTE-sialidase fusions, we also constructed and examined the activities of CD19- and EGFR-targeting BiKE-sialidase fusion proteins. To identify the optimal design for sialidase fused BiKEs, B. infantis sialidase was fused to the N-terminus or the C-terminus of BiKEs with (G4S)n linkers of different lengths (n=2, 3, or 4), respectively, as shown in FIG. 9. Two tumor antigens (TA), CD19 and EGFR, were selected for targeting as proof-of-concept. The nomenclature for each design of sialidase fused BiKEs were present in the figure. All BiKEs and sialidase fused BiKEs (SEQ ID NOs: 23-28) were fused with a 6x his tag at the C terminus for purification, and expressed in Expi293f cell system.

To compare the cytotoxicity of anti-CD19 or anti-EGFR BiKE and different design of sialidase fused BiKEs, CD19+ NALM6 cells (FIG. 10, left) and EGFR+ MDA-MB-231 cells (FIG. 10, right) were used as the target cells, and primary human NK cells were used as the effector cells. Human NK cells were purified from PBMCs by magnetic negative selection and then left overnight in culture medium in supplementation with 100 IU IL2 before killing assay. 1×10⁴ target cells were plated in 96-well plates, and BiKE, sialidase fused BiKEs (10 pm for anti-CD19 engagers and 100 pm for anti-EGFR engagers) or PBS were added to a final volume of 100 μl and treatments were pre-incubated with target cells for 30 min at 37° C. Next, 1×10⁴ human NK cells were added at effector to target (E/T) ratio of 1:1. The assay plate was incubated for 12 h and NK cell-mediated cytotoxicity was quantified by luciferin reporter assay. Three independent experimental replicates were shown. Two-way analysis of variance (ANOVA). According to the results shown here, we selected TAL4S design for the future experiments. Unless otherwise noted, BIKE-CD19-Sia and BIKE-EGFR-Sia refer to the TAL4S format (SEQ ID NOs: 26 and 28).

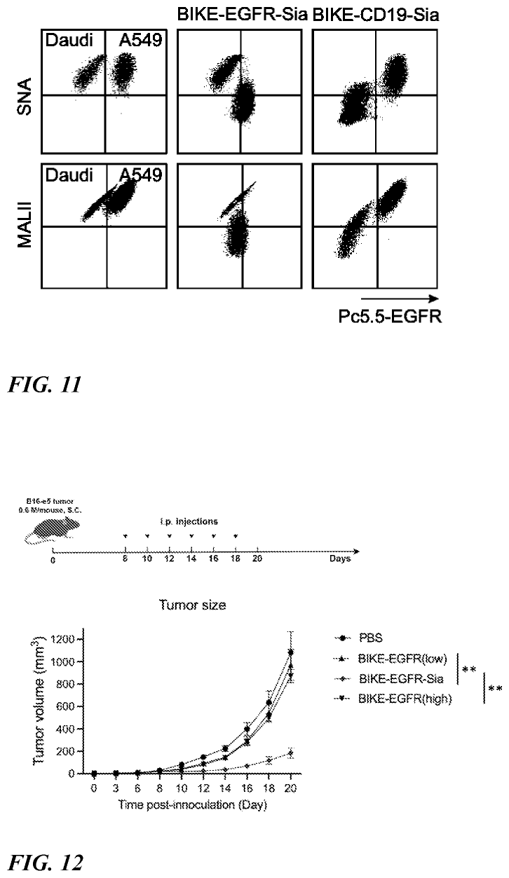

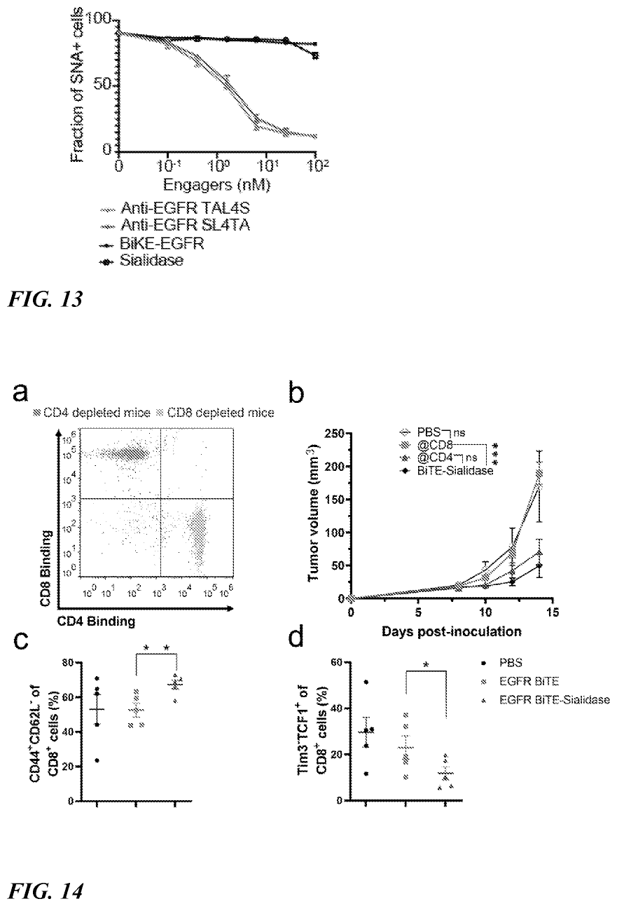

We compared the desialylation activity and selectivity of BiKE-CD19-Sia and BIKE-EGFR-Sia in a co-culture assay comprising CD19+ (Daudi) and EGFR+ (A549) cells, respectively. 1:1 ratio of daudi cells and A549 cells were treated with PBS, 10 nM BiKE-CD19-Sia or 10 nM BiKE-EGFR-Sia for 1h in serum-free medium, followed by staining with SNA-biotin (targets α2,6-linker sialoglycans) or MALII-biotin (targets α2,3-linker sialoglycans) in HBSS buffer for 30 min. Next, the cell mixtures were stained by streptavidin-APC and EGFR-Pc5.5. Representative flow cytometry dot plots from n=3 independent experiments were shown in FIG. 11. Additional data on desialylation activity of the BiKE sialidase fusion molecules are shown in FIG. 13.

Next, we compared anti-tumor functions of BiKE-EGFR-Sia and BIKE-EGFR in vivo (FIG. 12). B15E5 cells were inoculated subcutaneously into C57BL/6 mice, followed by intraperitoneal (i.p.) treatment with PBS, 5 μg BiKE-EGFR (low dose), 5 μg BiKE-EGFR (high dose), and 9 μg BiKE-EGFR-Sia when tumor size reaches 50 mm². The mice were injected i.p. every 2 days with PBS or engagers until the PBS-treated mice begin to reach a tumor burden requiring euthanasia. 6 independent experimental replicates were shown. Mean±s.e.m; **P<0.01, ****P<0.0001; Two-way analysis of variance.

### Example 11. BiTE mRNA Delivery Formulations

mRNAs for BiTE-sialidases were produced using in vitro translation (IVT). Briefly, the coding fragment of each protein was prepared cloned into pCS2+MT vectors with optimized 5′ (3′)-untranslated regions and poly A sequences. IVT reactions were performed following standard protocols but with N1-methylpseudouridine-5′-triphosphate replacing the typical uridine triphosphate. Finally, the mRNA was capped (Cap-1) using the vaccinia capping enzyme and 2′-O-methyltransferase New England Biolabs (NEB). RNA-loaded LNP formulations were formed using the ethanol dilution method.

All lipids with the following specified molar ratios were used:

0.43 mg ALC-0315=(4-hydroxybutyl) azanediyl)bis(hexane-6,1-diyl) bis(2-hexyldecanoate), 0.05 mg ALC-0159-2-[(polyethylene glycol)-2000]-N,N ditetradecylacetamide, 0.09 mg 1,2-Distearoyl-sn-glycero-3-phosphocholine (DSPC) 0.2 mg Cholesterol (46.3:9.4:42.7:1.6, Molar lipid ratios, %); or

SM-102 (heptadecan-9-yl 8-((2-hydroxyethyl) (6-oxo-6-(undecyloxy) hexyl) amino) octanoate} PEG2000-DMG=1-monomethoxypolyethyleneglycol-2,3-dimyristylglycerol with polyethylene glycol of average molecular weight 2000 1,2-Distearoyl-sn-glycero-3 phosphocholine (DSPC) Cholesterol (50:10:38.5:1.5, Molar lipid ratios, %).

All lipids with the specified molar ratios were dissolved in ethanol and mRNA was dissolved in 10 mM citrate buffer (pH 4.0). The two solutions were rapidly mixed at an aqueous to ethanol ratio of 3/1 by volume (3/1, aq./ethanol, vol./vol.) to satisfy a final weight ratio of 40/1 (total lipids/mRNA), then incubated for 10 min at room temperature. After LNP formation, the fresh LNP formulations were diluted with 1×PBS to 0.5 ng μl-1 mRNA (with a final ethanol concentration <5%) for in vitro assays and size detection. For in vivo experiments, the formulations were dialysed against 1×PBS for 2 h, and diluted with PBS for i.v. or s.c. injections.

### Example 12. Some Exemplified Methods and Materials

Cell lines and cell culturing: SK-BR-3 cells, MCF7 cells, PC3 cells, Raji cells, SKOV-3 cells, MDA-MB-435 cells, MDA-MB-231 cells, MDA-MB-468 cells, NALM-6, NK92MI were obtained from ATCC and they were cultured as suggested. B16-E5 cells were kindly gifted from Dr. Yangxin Fu. Expi293f cells were purchased from Thermo Fisher Scientific and cultured according to the protocol. For culturing of the isolated human PBMCs, AIM V™ Medium (Gibco™ 12055091) supplemented with 10% FBS was used. All cells were cultured in the incubator at 37° C. supplemented with 5% CO2.

General gene cloning procedures: The protein sequences of ScFv targeting human CD3, CD19, HER2 and PSMA were obtained from publicly available patents and the protein sequences were reverse-translated and codon-optimized to DNA sequences. All ScFv sequences were synthesized from IDT. The sequence of EGFR and murine CD3 binding ScFv were kindly provided by Dr. Yangxin Fu. The sequence of B. infantis sialidase was kindly gifted from George Peng Wang's lab. For the molecular cloning process, the difference sequences were assembled using NEBuilder HiFi DNA Assembly (New England BioLabs, E2621). For BiTE molecules, two separate ScFv sequences were connected by a GS linker such as GGGS (SEQ ID NO:1) or GGGGS (SEQ ID NO:38) linker. For the BiTE and sialidase fusion proteins, the sialidase sequence was conjugated to the BiTE sequence through a 2×GGGGS linker (SEQ ID NO:29).

Expression of BiTEs, B. infantis sialidase and BiTE-sialidase fusion proteins: All BiTEs, sialidase and BiTE-sialidase fusion proteins were fused with a 6×his tag at the C terminus for purification. For all BiTE and BiTE-sialidase fusion proteins, the expression was done in Expi293f cell system (Thermo Fisher Scientific). The transfection and handling of the cells were done according to the manufacturer's protocol. B. infantis sialidase was expressed in BL21 E. coli. For purification, all proteins were purified using Ni-NTA (nickel-nitrilotriacetic acid) resin from QIAGEN. After the incubation of Expi293 media supernatant with the Ni-NTA resin, the Nickle charged resin was washed with PBS and 20 mM imidazole. Proteins were eluted with 250 mM imidazole and were concentrated and buffer-exchanged to PBS before use. The concentration of all proteins was determined by Qubit Protein Quantification Assay (Thermo Fisher Scientific, Q33211).

Desialylation by B. infantis sialidase, 4D5 BiTE-sialidase fusion proteins and P-3Fax-Neu5Ac: For the removal of sialic acids by B. infantis sialidase or 4D5 BiTE-sialidase fusion proteins, 0.5 million cells were suspended in 100 mL DMEM without the serum. 1.5 mg sialidase or the fusion proteins were added in each sample and each sample was incubated at 37° C. for an hour. After the incubation, cells were washed twice by DPBS before they were used for killing experiments or staining. For the desialylation by inhibitor P-3Fax-Neu5Ac (R&D Systems, 117405-58-0), SK-BR-3 cells were cultured in T25 flask with the addition of 100 mM P-3Fax-Neu5Ac for three days.

Desialylation detection from SNA staining: For the SNA staining, 0.5 million cells with or without desialylation were suspended in 100 mL HBSS buffer (Sigma-Aldrich, H6648) supplemented with 5 mM CaCl2 and MgCl2. SNA-FITC was added at 1:200 and DAPI was added at 1:2000 to each sample and the mixture was incubated on ice for 30 min before washing twice with HBSS buffer. Samples were then analyzed by FACS. Desialylation was analyzed in DAPI negative live cell populations using Flowjo.

Human PBMC and T cell isolation: Human PBMCs were collected from blood samples of multiple healthy donors. Briefly, an equal amount of DPBS with 2 mM EDTA was used to dilute the blood samples. Then, the mixture was carefully added to Ficoll (Ficoll® Paque Plus, GE Healthcare, 17-1440-02) for gradient separation. After centrifuge at 650 g for 30 min with minimal acceleration and deceleration setting, the middle layer was collected and washed twice with DPBS supplemented with 2 mM EDTA. Further T cells isolation from human PBMCs was done with EasySep™ Human T Cell Isolation Kit (STEMCELL Technologies, 100-0695) according to the manufacturer's protocol.

Cell cytotoxicity measurement by lactate dehydrogenase (LDH) release: T cell cytotoxicity induced by BiTEs and BiTE-sialidase fusion proteins was measured by lactate dehydrogenase (LDH) release using CytoTox 96® Non-Radioactive Cytotoxicity Assay (Promega, G1780). tumor cells and hPBMCs (tumor cell:hPBMC=1:5) per well in 100 mL media were exposed to different treatments and incubated in 96 well plates at 37° C. (unless different ratio was specified elsewhere). After 24 hours of coincubation, 50 mL of media supernatant from each well was transferred to a new flat bottom 96 well plate and LDH release was measured using the supplier's protocol. Specific killing was calculated as suggested in the supplier's protocol with background subtraction and total lysis comparison.

Cytokine release and T cell surface activation marker measurement: For T cell cytokine release measurement, as with the cytotoxicity experiment, tumor cells and hPBMCs (tumor cell:hPBMC=1:5) were co-incubated per well in 96 well plates with different treatments in 100 mL media at 37° C. for 24 hours. Then, 20 mL of supernatant from each well was diluted in 100 mL DPBS and used for IFN-γ, IL-2 and TNFα measurement. The ELISA measurement was done by ELISA MAX™ Sets. IFN-γ, IL-2 and TNFα kits (BioLegend) and the experiments were done according to the manufacturer's protocol. The exact concentration was calculated from a standard curve. For the cell surface activation marker measurement, tumor cells and hPBMCs (tumor cell:hPBMC=1:5) were co-incubated per well in 12 well plates with different treatments in 1 mL media at 37° C. for 24 hours. Following incubation, cells from each well were resuspended and stained with anti-CD3-PE, anti-CD69-FITC, anti-CD25-APC or anti-CD107a-pacific blue (All from biolegend and were added at 1:200) for 30 min at 4° C. Cells were then washed twice with FACS buffer (PBS with 2.5% BSA) before being analyzed using flow cytometry. Data analysis and mean fluorescence intensity calculation were done by Flowjo. For the transcriptome analysis, 1.2 million hPBMCs and 0.1 million MDA-MB-231 cells were incubated together under the treatment of either 4 nM 4D5 BiTE or 4 nM 4D5 BiTE-sialidase (Three replicas for each condition). After 48 hours of incubation, the mixture was stained with DAPI and CD3 to sort out the T cell population. mRNA of T cell from each population was extracted by The Arcturus PicoPure RNA Isolation Kit (Thermo fisher). The mRNA samples were sent out to Novogene for sequencing and initial analyzing.

Flow cytometric analysis of Siglec-7 and Siglec-9 expression: Human PBMCs were collected from four healthy human donors. 0.5 million freshly isolated human PBMCs were suspended in 100 mL FACS buffer (PBS with 2.5% BSA) and each sample was stained with anti-CD3-PE. Each sample was also stained with either anti-Siglec-7-APC or anti-Siglec-9-APC (All from biolegend and were added at 1:200). After incubation for 30 min at 4° C., cells were washed twice with FACS buffer before being analyzed using flow cytometry. Positive population percentage of both Siglec-7 and Siglec-9-stained samples was analyzed by Flowjo. For T cells activated by BiTEs, 80000 tumor cells and 400000 hPBMCs were coincubated per well in a 12 well plates with or without the BiTEs and sialidase treatment in 1 mL media at 37° C. for 24 hours. Following incubation, cells were resuspended and stained as described earlier for Siglec-7 and Siglec-9 expression analysis.

Staining of human CD32 and actin for confocal imaging: Briefly, 0.4 million tumors cells were treated with 4 nM 4D5 BiTE or 4 nM 4D5 BiTE with 15 mg/ML sialidase in 100 mL DMEM without the serum for 1 hr at 37° C. After the incubation, all the samples were washed twice using PBS before incubating with 0.4 million hPBMCs in 500 ml PBS for 30 min at 37° C. Then all the cells were transferred in 1 ml PBS to the coverslips in 12 well plates and incubated at 37° C. for 30 min to let cells attach to the coverslip. 1 ml 4% PFA was added to each well and incubated with shaking for 20 min at room temperature (RT) for cell fixing, and then each well was washed twice with ice cold PBS. Washing took place at RT for 10 min with shaking. After fixation, 1 mL 0.1% PBS-Triton100 was added to each well for 10 min with shaking at RT to permeabilize the sample. PBST was used for washing for three times, each time with shaking at RT for 5 min. Next, 1 mL 2.5% FBS-PBST was used to block each sample for 50 min with shaking at RT. Then, anti-CD247 (CD3ζ) antibody (Sigma-Aldrich, 12-35-22-00) was diluted in FACs buffer at 1:200 and anti-actin antibody (Novus Biologicals, NBP267113) was diluted in 1:500. 500 mL of each diluted antibody was added to samples and incubated for an hour at RT with shaking. PBST was used for washing for three times before anti-rabbit 488 (Invitrogen, 35553) and anti-mouse 594 ((Invitrogen, A-11005) secondary antibody was diluted and used for staining at RT for 30 min with shaking. Finally, samples were washed three times and each coverslip was transferred to a glass slide with mounting oil. Fingernail oil was used to seal the coverslip. Samples were analyzed on a Zeiss LSM880 with a 63× oil lens (NA 1.4). The relative mean fluorescent intensity (MFI) of CD35 accumulation and relative contact area of IS was calculated by imageJ.

Cluster formation analysis: For the cluster formation experiments between SK-BR-3 cells and T cells. 0.5 million SK-BR-3 and 1 million hPMBCs were stained with CellTracker™ Green CMFDA (Thermo fisher) and PE anti-CD3 separately. After washing, they were incubated together under the treatment of the 50 nM 4D5 BiTE with or without the sialidase or 50 nM 4D5 BiTE-sialidase at 37° C. for 2 hours before the sample being analyzed by the FACS machine. The cluster experiment for the NALM-6 cells was of the same steps and settings except that NALM-6 GL cells carries GFP expression which doesn't need CellTracker staining. All results were analyzed by Flowjo.

RNA-sequencing analysis: Quality of raw sequencing reads was verified using FastQC (FastQC: A Quality Control Tool for High Throughput Sequence Data), which is available online. Reads were aligned to the genome and genic reads quantified using STAR version 2.7.0f (Dobin et al., Bioinformatics 29, 15-21, 2013) and Ensembl version 101 GRCm38 genome and transcriptome annotations. Normalization, differential expression analysis and principal component analysis were performed using R package DESeq2 v1.35.0. Heatmaps were constructed using R package ComplexHeatmap v2.12.0. R version 4.2.1 was used. Cytokine target expression analysis was performed using the python implementation of CytoSig (Jiang et al., Nature methods 18, 1181-1191, 2021). Gene set enrichment analysis was performed using GSEA (Subramanian et al., Proc. Natl. Acad. Sci. USA 102, 15545-15550, 2005).

Immunodeficient human tumor cell line xenograft mice model: All animal experiments were approved by the TSRI Animal Care and Use Committee. 15 NCG (6 weeks old male) mice (Charles Rivers Laboratories) were injected with 5×10⁶ human PBMCs (intraperitoneally) and 2.5×10⁶ SK-BR-3 cells (subcutaneously) on Day 0. On Day 6, mice were imaged by BLI and divided into groups based on similar tumor burden within each group. On Day 7, Three groups were intravenously (i.v) treated with PBS, 6 mg 4D5 BiTE, and 10 mg 4D5 BiTE-sialidase, respectively. Blood was collected from each mouse 5 hrs following BiTE administration and the serum IFN-γ level was measured using ELISA MAX™ (Biolengend). Drug treatment was continued twice a week, mouse received a second dose of 2× 10⁶ human PBMCs (intraperitoneally) and each on day 16. Tumor burden was imaged multiple times throughout the whole study process. For the BLI imaging, 200 mL 15 g/L D-Luciferin, Potassium Salt (GoldBio) was injected intraperitoneally in each mouse and mice were imaged by IVIS imaging system (PerkinElmer) after 10 mins. For the NALM-6 model, 20 NCG (6 weeks old male) mice (Charles Rivers Laboratories) were injected with 6×10⁶ human PBMCs (i.v) and 0.8×10⁶ NALM-6 cells (i.v) on Day 0. On Day 3, all mice were imaged and divided into four groups. 1.5 mg CD19 BiTE, 2.8 mg CD19 BiTE-sialidase, 4D5 BiTE-sialidase and PBS were injected into different groups, respectively. Tumor size was measured by BLI like described earlier until the death of the PBS control group.

B16-E5 syngeneic mice model: For the B16-E5 syngeneic mice model, 15 C57BL/6J mice (6 weeks old male) were injected with 0.6×10⁶ B16-E5 cells subcutaneously on day 0. On Day 8, tumor size was obtained by caliper measurement using the formula V=(W2×L)/2 and mice were divided into different groups. Intratumor injection of 0.5 mg EGFR BITE, 0.93 mg EGFR BiTE-sialidase and PBS were given to mice in different groups on Day 8, 12 and 14. Tumor size was recorded every two days until the mouse reached the endpoint of tumor size of 1000 mm³. For the tumor infiltrated lymphocytes profiling, 15 C57BL/6J mice (6 weeks old male) were also injected with 0.6×10⁶ B16-E5 cells subcutaneously on day 0. On Day 11, tumor size was measure and divided into three groups. 1.5 mg EGFR BiTE, 2.8 mg EGFR BiTE-sialidase and PBS were injected intratumorally into tumors in different groups. On Day 14, tumors were collected and tumor infiltrated lymphocytes from each tumor of different groups were stained with multiple markers for different populations within the CD45.2 lymphocytes for the profiling.

Statistical analysis: Unless specified elsewhere, results are shown using GraphPad Prism version 8.0.0 with standard error of the mean (SEM) as error bars, each dot represents a biological replicate. P values were calculated using the built-in data analysis function of Microsoft excel or GraphPad Prism8.

### Some Amino Acid Sequences Exemplified Herein

### Peptide Linkers:

(SEQ ID NO: 1)
GGGS
(SEQ ID NO: 2)
GGGSGGGS
(SEQ ID NO: 29)
GGGGSGGGGS
(SEQ ID NO: 30)
GGGGSGGGGSGGGGS
(SEQ ID NO: 31)
GGGGSGGGGSGGGGSGGGGS
(SEQ ID NO: 34)
SGGGG
(SEQ ID NO: 35)
SGGGGSGGGG
(SEQ ID NO: 36)
SGGGGSGGGGSGGGG
(SEQ ID NO: 37)
SGGGGSGGGGSGGGGSGGGG
(SEQ ID NO: 38)
GGGGS
4D5 scFv targeting HER2
(SEQ ID NO: 3)
HAMADIQMTQSPSSLSASVGDRVTITCRASQDVNTAVAWYQQKPGKAPKLLIYSAS
FLYSGVPSRFSGSRSGTDFTLTISSLQPEDFATYYCQQHYTTPPTFGQGTKVEIKGAS
GGGGSGGGGSGGGGSSEVQLVESGGGLVQPGGSLRLSCAASGFNIKDTYIHWVRQA
PGKGLEWVARIYPTNGYTRYADSVKGRFTISADTSKNTAYLQMNSLRAEDTAVYY
CSRWGGDGFYAMDYWGQGTLVTVSSGS
Anti CD3 scFv
(SEQ ID NO: 4)
DIKLQQSGAELARPGASVKMSCKTSGYTFTRYTMHWVKQRPGQGLEWIGYINPSRGYTNY
NQKFKDKATLTTDKSSSTAYMQLSSLTSEDSAVYYCARYYDDHYCLDYWGQGTTLTVSSVE
GGSGGSGGSGGSGGVDDIQLTQSPAIMSASPGEKVTMTCRASSSVSYMNWYQQKSGTSPK
RWIYDTSKVASGVPYRFSGSGSGTSYSLTISSMEAEDAATYYCQQWSSNPLTFGAGTKLELK
Bifidobacterium longum subspecies infantis (B. infantis) sialidase
(SEQ ID NO: 5)
MTENGMMNTNNTVCGANHDGAMSLAAPGDYGVACYRIPALAEAPNGWILAAFDA
RPHNCQDAPQANSIVQRISKDGGRSFEPQHVVAAGHDGVDKYGYSDPSYVVDRQT
GEVFLFFVKSYDAGFGTSQAGVDPSAREVLQAAVTSSIDNGVTWSEPRIITADITNSE
SWISRFASSGAGIQLTYGEHAGRLIQQYTIKELDGRYRAVSVFSDDHGATWHAGTPV
GDHMDENKVVELSDGRVMLNSRSSDGNGCRYVAISRDGGATYGPVIRETQLPDPEN
NAQIARAFPDAPEGSAQAKVLLYSSSSPSDRIDGLVRVSIDDGKTWSAGRRFTTGPM
AYSVIAALSHKAGGGYGLLYEGDNNNIMYTRISLDWLNGQLNVDGIGGFPLSGEGG
C
4D5-CD3 BITE (4D5 scFv sequence underlined; anti-CD3 sequence italicized)
(SEQ ID NO: 6)
HAMADIQMTQSPSSLSASVGDRVTITCRASQDVNTAVAWYQQKPGKAPKLLIYSAS
FLYSGVPSRFSGSRSGTDFTLTISSLQPEDFATYYCQQHYTTPPTFGQGTKVEIKGAS
GGGGSGGGGSGGGGSSEVQLVESGGGLVQPGGSLRLSCAASGFNIKDTYIHWVRQA
PGKGLEWVARIYPTNGYTRYADSVKGRFTISADTSKNTAYLQMNSLRAEDTAVYY
CSRWGGDGFYAMDYWGQGTLVTVSSGS
DIKLQQSGAELARPGASVKMSCKTSGYTFTRYTMHWVKQRPGQGLEWIGYINPSRGYTNY
NQKFKDKATLTTDKSSSTAYMQLSSLTSEDSAVYYCARYYDDHYCLDYWGQGTTLTVSSV
EGGSGGSGGSGGSGGVDDIQLTQSPAIMSASPGEKVTMTCRASSSVSYMNWYQQKSGTS
PKRWIYDTSKVASGVPYRFSGSGSGTSYSLTISSMEAEDAATYYCQQWSSNPLTFGAGTKL
ELK
4D5-CD3 BiTE-sialidase fusion (linker sequence connecting the BiTE and
sialidase is bolded and italicized)
(SEQ ID NO: 7)
HAMADIQMTQSPSSLSASVGDRVTITCRASQDVNTAVAWYQQKPGKAPKLLIYSAS
FLYSGVPSRFSGSRSGTDFTLTISSLQPEDFATYYCQQHYTTPPTFGQGTKVEIKGAS
GGGGSGGGGSGGGGSSEVQLVESGGGLVQPGGSLRLSCAASGFNIKDTYIHWVRQA
PGKGLEWVARIYPTNGYTRYADSVKGRFTISADTSKNTAYLQMNSLRAEDTAVYY
CSRWGGDGFYAMDYWGQGTLVTVSSGS
DIKLQQSGAELARPGASVKMSCKTSGYTFTRYTMHWVKQRPGQGLEWIGYINPSRGYTNY
NQKFKDKATLTTDKSSSTAYMQLSSLTSEDSAVYYCARYYDDHYCLDYWGQGTTLTVSSV
EGGSGGSGGSGGSGGVDDIQLTQSPAIMSASPGEKVTMTCRASSSVSYMNWYQQKSGTS
PKRWIYDTSKVASGVPYRFSGSGSGTSYSLTISSMEAEDAATYYCQQWSSNPLTFGAGTKL
ELKGGGSGGGS
MTENGMMNTNNTVCGANHDGAMSLAAPGDYGVACYRIPALAEAPNGWILAAFDA
RPHNCQDAPQANSIVQRISKDGGRSFEPQHVVAAGHDGVDKYGYSDPSYVVDRQT
GEVFLFFVKSYDAGFGTSQAGVDPSAREVLQAAVTSSIDNGVTWSEPRIITADITNSE
SWISRFASSGAGIQLTYGEHAGRLIQQYTIKELDGRYRAVSVFSDDHGATWHAGTP
VGDHMDENKVVELSDGRVMLNSRSSDGNGCRYVAISRDGGATYGPVIRETQLPDP
ENNAQIARAFPDAPEGSAQAKVLLYSSSSPSDRIDGLVRVSIDDGKTWSAGRRFTTG
PMAYSVIAALSHKAGGGYGLLYEGDNNNIMYTRISLDWLNGQLNVDGIGGFPLSGE
GGC
Sialidase 4D5-BiTE fusion (linker sequence connecting the sialidase and the
BiTE is bolded and italicized)
(SEQ ID NO: 8)
MTENGMMNTNNTVCGANHDGAMSLAAPGDYGVACYRIPALAEAPNGWILAAFDA
RPHNCQDAPQANSIVQRISKDGGRSFEPQHVVAAGHDGVDKYGYSDPSYVVDRQT
GEVFLFFVKSYDAGFGTSQAGVDPSAREVLQAAVTSSIDNGVTWSEPRIITADITNSE
SWISRFASSGAGIQLTYGEHAGRLIQQYTIKELDGRYRAVSVFSDDHGATWHAGTP
VGDHMDENKVVELSDGRVMLNSRSSDGNGCRYVAISRDGGATYGPVIRETQLPDP
ENNAQIARAFPDAPEGSAQAKVLLYSSSSPSDRIDGLVRVSIDDGKTWSAGRRFTTG
PMAYSVIAALSHKAGGGYGLLYEGDNNNIMYTRISLDWLNGQLNVDGIGGFPLSGE
GGCGGGSGGGS-
HAMADIQMTQSPSSLSASVGDRVTITCRASQDVNTAVAWYQQKPGKAPKLLIYSAS
FLYSGVPSRFSGSRSGTDFTLTISSLQPEDFATYYCQQHYTTPPTFGQGTKVEIKGAS
GGGGSGGGGSGGGGSSEVQLVESGGGLVQPGGSLRLSCAASGFNIKDTYIHWVRQA
PGKGLEWVARIYPTNGYTRYADSVKGRFTISADTSKNTAYLQMNSLRAEDTAVYY
CSRWGGDGFYAMDYWGQGTLVTVSSGS-
DIKLQQSGAELARPGASVKMSCKTSGYTFTRYTMHWVKQRPGQGLEWIGYINPSRGYTNY
NQKFKDKATLTTDKSSSTAYMQLSSLTSEDSAVYYCARYYDDHYCLDYWGQGTTLTVSSV
EGGSGGSGGSGGSGGVDDIQLTQSPAIMSASPGEKVTMTCRASSSVSYMNWYQQKSGTS
PKRWIYDTSKVASGVPYRFSGSGSGTSYSLTISSMEAEDAATYYCQQWSSNPLTFGAGTKL
ELK
PSMA-targeting scFv
(SEQ ID NO: 9)
EVQLQQSGPELVKPGTSVRISCKTSGYTFTEYTIHWVKQSHGKSLEWIGNINPNNGG
TTYNQKFEDKATLTVDKSSSTAYMELRSLTSEDSAVYYCAAGWNFDYWGQGTTLTV
SSGGGGSGGGGSGGGGSDIVMTQSHKFMSTSVGDRVSIICKASQDVGTAVDWYQQK
PGQSPKLLIYWASTRHTGVPDRFTGSGSGTDFTLTITNVQSEDLADYFCQQYNSYPLT
FGAGTMLDLK
PSMA-CD3 BiTE (anti-PSMA scFv underlined; anti-CD3 italicized; GS linker
bolded and italicized)
(SEQ ID NO: 10)
EVQLQQSGPELVKPGTSVRISCKTSGYTFTEYTIHWVKQSHGKSLEWIGNINPNNGG
TTYNQKFEDKATLTVDKSSSTAYMELRSLTSEDSAVYYCAAGWNFDYWGQGTTLTV
SSGGGGSGGGGSGGGGSDIVMTQSHKFMSTSVGDRVSIICKASQDVGTAVDWYQQK
PGQSPKLLIYWASTRHTGVPDRFTGSGSGTDFTLTITNVQSEDLADYFCQQYNSYPLT
FGAGTMLDLKGGGS-
DIKLQQSGAELARPGASVKMSCKTSGYTFTRYTMHWVKQRPGQGLEWIGYINPSRGYTNY
NQKFKDKATLTTDKSSSTAYMQLSSLTSEDSAVYYCARYYDDHYCLDYWGQGTTLTVSSVE
GGSGGSGGSGGSGGVDDIQLTQSPAIMSASPGEKVTMTCRASSSVSYMNWYQQKSGTSPK
RWIYDTSKVASGVPYRFSGSGSGTSYSLTISSMEAEDAATYYCQQWSSNPLTFGAGTKLELK
PSMA-CD3 BiTE sialidase fusion (linker sequence connecting the BiTE and the
sialidase is underlined and italicized)
(SEQ ID NO: 11)
EVQLQQSGPELVKPGTSVRISCKTSGYTFTEYTIHWVKQSHGKSLEWIGNINPNNGG
TTYNQKFEDKATLTVDKSSSTAYMELRSLTSEDSAVYYCAAGWNFDYWGQGTTLT
VSSGGGGSGGGGSGGGGSDIVMTQSHKFMSTSVGDRVSIICKASQDVGTAVDWYQ
QKPGQSPKLLIYWASTRHTGVPDRFTGSGSGTDFTLTITNVQSEDLADYFCQQYNSY
PLTFGAGTMLDLKGGGS-
DIKLQQSGAELARPGASVKMSCKTSGYTFTRYTMHWVKQRPGQGLEWIGYINPSRGYTNY
NQKFKDKATLTTDKSSSTAYMQLSSLTSEDSAVYYCARYYDDHYCLDYWGQGTTLTVSSV
EGGSGGSGGSGGSGGVDDIQLTQSPAIMSASPGEKVTMTCRASSSVSYMNWYQQKSGTS
PKRWIYDTSKVASGVPYRFSGSGSGTSYSLTISSMEAEDAATYYCQQWSSNPLTFGAGTKL
ELKGGGSGGGS-
MTENGMMNTNNTVCGANHDGAMSLAAPGDYGVACYRIPALAEAPNGWILAAFDA
RPHNCQDAPQANSIVQRISKDGGRSFEPQHVVAAGHDGVDKYGYSDPSYVVDRQT
GEVFLFFVKSYDAGFGTSQAGVDPSAREVLQAAVTSSIDNGVTWSEPRIITADITNSE
SWISRFASSGAGIQLTYGEHAGRLIQQYTIKELDGRYRAVSVFSDDHGATWHAGTP
VGDHMDENKVVELSDGRVMLNSRSSDGNGCRYVAISRDGGATYGPVIRETQLPDP
ENNAQIARAFPDAPEGSAQAKVLLYSSSSPSDRIDGLVRVSIDDGKTWSAGRRFTTG
PMAYSVIAALSHKAGGGYGLLYEGDNNNIMYTRISLDWLNGQLNVDGIGGFPLSGE
GGC
Blinatumomab (CD19 BiTE)
(SEQ ID NO: 12)
DIQLTQSPASLAVSLGQRATISCKASQSVDYDGDSYLNWYQQIPGQPPKLLIYDASNL
VSGIPPRFSGSGSGTDFTLNIHPVEKVDAATYHCQQSTEDPWTFGGGTKLEIKGGGGS
GGGGSGGGGSQVQLQQSGAELVRPGSSVKISCKASGYAFSSYWMNWVKQRPGQGL
EWIGQIWPGDGDTNYNGKFKGKATLTADESSSTAYMQLSSLASEDSAVYFCARRETT
TVGRYYYAMDYWGQGTTVTVSSGGGGSDIKLQQSGAELARPGASVKMSCKTSGYT
FTRYTMHWVKQRPGQGLEWIGYINPSRGYTNYNQKFKDKATLTTDKSSSTAYMQLS
SLTSEDSAVYYCARYYDDHYCLDYWGQGTTLTVSSVEGGSGGSGGSGGSGGVDDIQ
LTQSPAIMSASPGEKVTMTCRASSSVSYMNWYQQKSGTSPKRWIYDTSKVASGVPY
RFSGSGSGTSYSLTISSMEAEDAATYYCQQWSSNPLTFGAGTKLELK
Blinatumomab-sialidase fusion (CD19 BiTE-sialidase)
(SEQ ID NO: 13)
DIQLTQSPASLAVSLGQRATISCKASQSVDYDGDSYLNWYQQIPGQPPKLLIYDASN
LVSGIPPRFSGSGSGTDFTLNIHPVEKVDAATYHCQQSTEDPWTFGGGTKLEIKGGG
GSGGGGSGGGGSQVQLQQSGAELVRPGSSVKISCKASGYAFSSYWMNWVKQRPGQ
GLEWIGQIWPGDGDTNYNGKFKGKATLTADESSSTAYMQLSSLASEDSAVYFCARR
ETTTVGRYYYAMDYWGQGTTVTVSSGGGGSDIKLQQSGAELARPGASVKMSCKTS
GYTFTRYTMHWVKQRPGQGLEWIGYINPSRGYTNYNQKFKDKATLTTDKSSSTAY
MQLSSLTSEDSAVYYCARYYDDHYCLDYWGQGTTLTVSSVEGGSGGSGGSGGSGG
VDDIQLTQSPAIMSASPGEKVTMTCRASSSVSYMNWYQQKSGTSPKRWIYDTSKVA
SGVPYRFSGSGSGTSYSLTISSMEAEDAATYYCQQWSSNPLTFGAGTKLELKGGGSG
GGS-
MTENGMMNTNNTVCGANHDGAMSLAAPGDYGVACYRIPALAEAPNGWILAAFDA
RPHNCQDAPQANSIVQRISKDGGRSFEPQHVVAAGHDGVDKYGYSDPSYVVDRQT
GEVFLFFVKSYDAGFGTSQAGVDPSAREVLQAAVTSSIDNGVTWSEPRIITADITNSE
SWISRFASSGAGIQLTYGEHAGRLIQQYTIKELDGRYRAVSVFSDDHGATWHAGTP
VGDHMDENKVVELSDGRVMLNSRSSDGNGCRYVAISRDGGATYGPVIRETQLPDP
ENNAQIARAFPDAPEGSAQAKVLLYSSSSPSDRIDGLVRVSIDDGKTWSAGRRFTTG
PMAYSVIAALSHKAGGGYGLLYEGDNNNIMYTRISLDWLNGQLNVDGIGGFPLSGE
GGC
5E5-BiTE (anti Tn-MUC1)
(SEQ ID NO: 14)
QVQLQQSDAELVKPGSSVKISCKASGYTFTDHAIHWVKQKPEQGLEWIGHFSPGNT
DIKYNDKFKGKATLTVDRSSSTAYMQLNSLTSEDSAVYFCKTSTFFFDYWGQGTTL
TVSSGGGGSGGGGSGGGGSDIVMTQSPSSLTVTAGEKVTMICKSSQSLLNSGDQKN
YLTWYQQKPGQPPKLLIFWASTRESGVPDRFTGSGSGTDFTLTISSVQAEDLAVYYC
QNDYSYPLTFGAGTKLELKGGGSDIKLQQSGAELARPGASVKMSCKTSGYTFTRYT
MHWVKQRPGQGLEWIGYINPSRGYTNYNQKFKDKATLTTDKSSSTAYMQLSSLTS
EDSAVYYCARYYDDHYCLDYWGQGTTLTVSSVEGGSGGSGGSGGSGGVDDIQLTQ
SPAIMSASPGEKVTMTCRASSSVSYMNWYQQKSGTSPKRWIYDTSKVASGVPYRFS
GSGSGTSYSLTISSMEAEDAATYYCQQWSSNPLTFGAGTKLELK
5E5 BiTE-sialidase fusion
(SEQ ID NO: 15)
QVQLQQSDAELVKPGSSVKISCKASGYTFTDHAIHWVKQKPEQGLEWIGHFSPGNT
DIKYNDKFKGKATLTVDRSSSTAYMQLNSLTSEDSAVYFCKTSTFFFDYWGQGTTL
TVSSGGGGSGGGGSGGGGSDIVMTQSPSSLTVTAGEKVTMICKSSQSLLNSGDQKN
YLTWYQQKPGQPPKLLIFWASTRESGVPDRFTGSGSGTDFTLTISSVQAEDLAVYYC
QNDYSYPLTFGAGTKLELKGGGSDIKLQQSGAELARPGASVKMSCKTSGYTFTRYT
MHWVKQRPGQGLEWIGYINPSRGYTNYNQKFKDKATLTTDKSSSTAYMQLSSLTS
EDSAVYYCARYYDDHYCLDYWGQGTTLTVSSVEGGSGGSGGSGGSGGVDDIQLTQ
SPAIMSASPGEKVTMTCRASSSVSYMNWYQQKSGTSPKRWIYDTSKVASGVPYRFS
GSGSGTSYSLTISSMEAEDAATYYCQQWSSNPLTFGAGTKLELKGGGSGGGS
MTENGMMNTNNTVCGANHDGAMSLAAPGDYGVACYRIPALAEAPNGWILAAFDA
RPHNCQDAPQANSIVQRISKDGGRSFEPQHVVAAGHDGVDKYGYSDPSYVVDRQT
GEVFLFFVKSYDAGFGTSQAGVDPSAREVLQAAVTSSIDNGVTWSEPRIITADITNSE
SWISRFASSGAGIQLTYGEHAGRLIQQYTIKELDGRYRAVSVFSDDHGATWHAGTP
VGDHMDENKVVELSDGRVMLNSRSSDGNGCRYVAISRDGGATYGPVIRETQLPDP
ENNAQIARAFPDAPEGSAQAKVLLYSSSSPSDRIDGLVRVSIDDGKTWSAGRRFTTG
PMAYSVIAALSHKAGGGYGLLYEGDNNNIMYTRISLDWLNGQLNVDGIGGFPLSGE
GGC

### Human Sialidase Sequences (SEQ ID NOs: 16-22)

Human NEU 1 (SEQ ID NO: 16):
MTGERPSTAL PDRRWGPRIL GFWGGCRVWV FAAIFLLLSL AASWSKAEND
FGLVQPLVTM EQLLWVSGRQ IGSVDTFRIP LITATPRGTL LAFAEARKMS
SSDEGAKFIA LRRSMDQGST WSPTAFIVND GDVPDGLNLG AVVSDVETGV
VFLFYSLCAH KAGCQVASTM LVWSKDDGVS WSTPRNLSLD IGTEVFAPGP
GSGIQKQREP RKGRLIVCGH GTLERDGVFC LLSDDHGASW RYGSGVSGIP
YGQPKQENDF NPDECQPYEL PDGSVVINAR NQNNYHCHCR IVLRSYDACD
TLRPRDVTFD PELVDPVVAA GAVVISSGIV FFSNPAHPEF RVNLTLRWSF
SNGTSWRKET VQLWPGPSGY SSLATLEGSM DGEEQAPQLY VLYEKGRNHY
TESISVAKIS VYGTL
Human NEU 2
(SEQ ID NO: 17)
MASLPVLQKE SVFQSGAHAY RIPALLYLPG QQSLLAFAEQ RASKKDEHAE
LIVLRRGDYD APTHQVQWQA QEVVAQARLD GHRSMNPCPL YDAQTGTLFL
FFIAIPGQVT EQQQLQTRAN VTRLCQVIST DHGRTWSSPR DLTDAAIGPA
YREWSTFAVG PGHCLQLHDR ARSLVVPAYA YRKLHPIQRP IPSAFCFLSH
DHGRTWARGH FVAQDTLECQ VAEVETGEQR VVTLNARSHL RARVQAQSTN
DGLDFQESQL VKKLVEPPPQ GCQGSVISFP SPRSGPGSPA QWLLYTHPTH
SWQRADLGAY LNPRPPAPEA WSEPVLLAKG SCAYSDLQSM GTGPDGSPLF
GCLYEANDYE EIVFLMFTLK QAFPAEYLPQ
Human NEU 3, isoform 1
(SEQ ID NO: 18)
MEEVTTCSFN SPLFRQEDDR GITYRIPALL YIPPTHTFLA FAEKRSTRRD
EDALHLVLRR GLRIGQLVQW GPLKPLMEAT LPGHRTMNPC PVWEQKSGCV
FLFFICVRGH VTERQQIVSG RNAARLCFIY SQDAGCSWSE VRDLTEEVIG
SELKHWATFA VGPGHGIQLQ SGRLVIPAYT YYIPSWFFCF QLPCKTRPHS
LMIYSDDLGV TWHHGRLIRP MVTVECEVAE VTGRAGHPVL YCSARTPNRC
RAEALSTDHG EGFQRLALSR QLCEPPHGCQ GSVVSFRPLE IPHRCQDSSS
KDAPTIQQSS PGSSLRLEEE AGTPSESWLL YSHPTSRKQR VDLGIYLNQT
PLEAACWSRP WILHCGPCGY SDLAALEEEG LFGCLFECGT KQECEQIAFR
LFTHREILSH LQGDCTSPGR NPSQFKSN
Human NEU 3, isoform 2
(SEQ ID NO: 19)
MRPADLPPRP MEESPASSSA PTETEEPGSS AEVMEEVTTC SFNSPLFRQE
DDRGITYRIP ALLYIPPTHT FLAFAEKRST RRDEDALHLV LRRGLRIGQL
VQWGPLKPLM EATLPGHRTM NPCPVWEQKS GCVFLFFICV RGHVTERQQI
VSGRNAARLC FIYSQDAGCS WSEVRDLTEE VIGSELKHWA TFAVGPGHGI
QLQSGRLVIP AYTYYIPSWF FCFQLPCKTR PHSLMIYSDD LGVTWHHGRL
IRPMVTVECE VAEVTGRAGH PVLYCSARTP NRCRAEALST DHGEGFQRLA
LSRQLCEPPH GCQGSVVSFR PLEIPHRCQD SSSKDAPTIQ QSSPGSSLRL
EEEAGTPSES WLLYSHPTSR KQRVDLGIYL NQTPLEAACW SRPWILHCGP
CGYSDLAALE EEGLFGCLFE CGTKQECEQI AFRLFTHREI LSHLQGDCTS
PGRNPSQFKS N
Human NEU 4, isoform 1
(SEQ ID NO: 20)
MMSSAAFPRW LSMGVPRTPS RTVLFERERT GLTYRVPSLL PVPPGPTLLA
FVEQRLSPDD SHAHRLVLRR GTLAGGSVRW GALHVLGTAA LAEHRSMNPC
PVHDAGTGTV FLFFIAVLGH TPEAVQIATG RNAARLCCVA SRDAGLSWGS
ARDLTEEAIG GAVQDWATFA VGPGHGVQLP SGRLLVPAYT YRVDRRECFG
KICRTSPHSF AFYSDDHGRT WRCGGLVPNL RSGECQLAAV DGGQAGSFLY
CNARSPLGSR VQALSTDEGT SFLPAERVAS LPETAWGCQG SIVGFPAPAP
NRPRDDSWSV GPGSPLQPPL LGPGVHEPPE EAAVDPRGGQ VPGGPFSRLQ
PRGDGPRQPG PRPGVSGDVG SWTLALPMPF AAPPQSPTWL LYSHPVGRRA
RLHMGIRLSQ SPLDPRSWTE PWVIYEGPSG YSDLASIGPA PEGGLVFACL
YESGARTSYD EISFCTFSLR EVLENVPASP KPPNLGDKPR GCCWPS
Human NEU 4, isoform 2
(SEQ ID NO: 21)
MMSSAAFPRW LQSMGVPRTP SRTVLFERER TGLTYRVPSL LPVPPGPTLL
AFVEQRLSPD DSHAHRLVLR RGTLAGGSVR WGALHVLGTA ALAEHRSMNP
CPVHDAGTGT VFLFFIAVLG HTPEAVQIAT GRNAARLCCV ASRDAGLSWG
SARDLTEEAI GGAVQDWATF AVGPGHGVQL PSGRLLVPAY TYRVDRRECF
GKICRTSPHS FAFYSDDHGR TWRCGGLVPN LRSGECQLAA VDGGQAGSFL
YCNARSPLGS RVQALSTDEG TSFLPAERVA SLPETAWGCQ GSIVGFPAPA
PNRPRDDSWS VGPGSPLQPP LLGPGVHEPP EEAAVDPRGG QVPGGPFSRL
QPRGDGPRQP GPRPGVSGDV GSWTLALPMP FAAPPQSPTW LLYSHPVGRR
ARLHMGIRLS QSPLDPRSWT EPWVIYEGPS GYSDLASIGP APEGGLVFAC
LYESGARTSY DEISFCTFSL REVLENVPAS PKPPNLGDKP RGCCWPS
Human NEU 4, isoform 3
(SEQ ID NO: 22)
MGVPRTPSRT VLFERERTGL TYRVPSLLPV PPGPTLLAFV EQRLSPDDSH
AHRLVLRRGT LAGGSVRWGA LHVLGTAALA EHRSMNPCPV HDAGTGTVFL
FFIAVLGHTP EAVQIATGRN AARLCCVASR DAGLSWGSAR DLTEEAIGGA
VQDWATFAVG PGHGVQLPSG RLLVPAYTYR VDRRECFGKI CRTSPHSFAF
YSDDHGRTWR CGGLVPNLRS GECQLAAVDG GQAGSFLYCN ARSPLGSRVQ
ALSTDEGTSF LPAERVASLP ETAWGCQGSI VGFPAPAPNR PRDDSWSVGP
GSPLQPPLLG PGVHEPPEEA AVDPRGGQVP GGPFSRLQPR GDGPRQPGPR
PGVSGDVGSW TLALPMPFAA PPQSPTWLLY SHPVGRRARL HMGIRLSQSP
LDPRSWTEPW VIYEGPSGYS DLASIGPAPE GGLVFACLYE SGARTSYDEI
SFCTFSLREV LENVPASPKP PNLGDKPRGC CWPS
CD19-CD16A BIKE
(SEQ ID NO: 32)
DIQLTQSPASLAVSLGQRATISCKASQSVDYDGDSYLNWYQQIPGQPPKLLIYDASN
LVSGIPPRFSGSGSGTDFTLNIHPVEKVDAATYHCQQSTEDPWTFGGGTKLEIKGGG
GSGGGGSGGGGSQVQLQQSGAELVRPGSSVKISCKASGYAFSSYWMNWVKQRPGQ
GLEWIGQIWPGDGDTNYNGKFKGKATLTADESSSTAYMQLSSLASEDSAVYFCARR
ETTTVGRYYYAMDYWGQGTTVTVSSGGGGSQVQLVQSGAEVKKPGESLKVSCKA
SGYTFTSYYMHWVRQAPGQGLEWMGIINPSGGSTSYAQKFQGRVTMTRDTSTSTV
YMELSSLRSEDTAVYYCARGSAYYYDFADYWGQGTLVTVSSGGGGSGGGGSGGG
GSSYVLTQPSSVSVAPGQTATISCGGHNIGSKNVHWYQQRPGQSPVLVIYQDNKRPS
GIPERFSGSNSGNTATLTISGTQAMDEADYYCQVWDNYSVLFGGGTKLTVL
EGFR-CD16A BIKE
(SEQ ID NO: 33)
QVQLQESGPGLVKPSETLSLTCTVSGGSVSSGSYYWSWIRQPPGKGLEWIGYIYYSG
STNYNPSLKSRVTISVDTSKNQFSLKLSSVTAADTAVYYCARNPISIPAFDIWGQGTM
VTVSSGGGGSGGGGSGGGGSQPVLTQPPSVSVAPGKTARITCGGNNIGSKSVHWYQ
QKPGQAPVLVIYYDSDRPSGIPERFSGSNSGNTATLTISRVEAGDEADYYCQVWDTS
SDHVLFGGGTKLTVLGGGGSQVQLVQSGAEVKKPGESLKVSCKASGYTFTSYYMH
WVRQAPGQGLEWMGIINPSGGSTSYAQKFQGRVTMTRDTSTSTVYMELSSLRSEDT
AVYYCARGSAYYYDFADYWGQGTLVTVSS
Anti-CD19 BiKE-Sialidase fusion TAL2S
(SEQ ID NO: 23)
DIQLTQSPASLAVSLGQRATISCKASQSVDYDGDSYLNWYQQIPGQPPKLLIYDASN
LVSGIPPRFSGSGSGTDFTLNIHPVEKVDAATYHCQQSTEDPWTFGGGTKLEIKGGG
GSGGGGSGGGGSQVQLQQSGAELVRPGSSVKISCKASGYAFSSYWMNWVKQRPGQ
GLEWIGQIWPGDGDTNYNGKFKGKATLTADESSSTAYMQLSSLASEDSAVYFCARR
ETTTVGRYYYAMDYWGQGTTVTVSSGGGGSQVQLVQSGAEVKKPGESLKVSCKA
SGYTFTSYYMHWVRQAPGQGLEWMGIINPSGGSTSYAQKFQGRVTMTRDTSTSTV
YMELSSLRSEDTAVYYCARGSAYYYDFADYWGQGTLVTVSSGGGGSGGGGSGGG
GSSYVLTQPSSVSVAPGQTATISCGGHNIGSKNVHWYQQRPGQSPVLVIYQDNKRPS
GIPERFSGSNSGNTATLTISGTQAMDEADYYCQVWDNYSVLFGGGTKLTVLGGGGS
GGGGS
MTENGMMNTNNTVCGANHDGAMSLAAPGDYGVACYRIPALAEAPNGWILAAFDA
RPHNCQDAPQANSIVQRISKDGGRSFEPQHVVAAGHDGVDKYGYSDPSYVVDRQT
GEVFLFFVKSYDAGFGTSQAGVDPSAREVLQAAVTSSIDNGVTWSEPRIITADITNSE
SWISRFASSGAGIQLTYGEHAGRLIQQYTIKELDGRYRAVSVFSDDHGATWHAGTP
VGDHMDENKVVELSDGRVMLNSRSSDGNGCRYVAISRDGGATYGPVIRETQLPDP
ENNAQIARAFPDAPEGSAQAKVLLYSSSSPSDRIDGLVRVSIDDGKTWSAGRRFTTG
PMAYSVIAALSHKAGGGYGLLYEGDNNNIMYTRISLDWLNGQLNVDGIGGFPLSGE
GGC
Anti-EGFR BiKE-Sialidase fusion TAL2S
(SEQ ID NO: 24)
QVQLQESGPGLVKPSETLSLTCTVSGGSVSSGSYYWSWIRQPPGKGLEWIGYIYYSG
STNYNPSLKSRVTISVDTSKNQFSLKLSSVTAADTAVYYCARNPISIPAFDIWGQGTM
VTVSSGGGGSGGGGSGGGGSQPVLTQPPSVSVAPGKTARITCGGNNIGSKSVHWYQ
QKPGQAPVLVIYYDSDRPSGIPERFSGSNSGNTATLTISRVEAGDEADYYCQVWDTS
SDHVLFGGGTKLTVLGGGGSQVQLVQSGAEVKKPGESLKVSCKASGYTFTSYYMH
WVRQAPGQGLEWMGIINPSGGSTSYAQKFQGRVTMTRDTSTSTVYMELSSLRSEDT
AVYYCARGSAYYYDFADYWGQGTLVTVSSGGGGSGGGGSGGGGSSYVLTQPSSVS
VAPGQTATISCGGHNIGSKNVHWYQQRPGQSPVLVIYQDNKRPSGIPERFSGSNSGN
TATLTISGTQAMDEADYYCQVWDNYSVLFGGGTKLTVLGGGGSGGGGS
MTENGMMNTNNTVCGANHDGAMSLAAPGDYGVACYRIPALAEAPNGWILAAFDA
RPHNCQDAPQANSIVQRISKDGGRSFEPQHVVAAGHDGVDKYGYSDPSYVVDRQT
GEVFLFFVKSYDAGFGTSQAGVDPSAREVLQAAVTSSIDNGVTWSEPRIITADITNSE
SWISRFASSGAGIQLTYGEHAGRLIQQYTIKELDGRYRAVSVFSDDHGATWHAGTP
VGDHMDENKVVELSDGRVMLNSRSSDGNGCRYVAISRDGGATYGPVIRETQLPDP
ENNAQIARAFPDAPEGSAQAKVLLYSSSSPSDRIDGLVRVSIDDGKTWSAGRRFTTG
PMAYSVIAALSHKAGGGYGLLYEGDNNNIMYTRISLDWLNGQLNVDGIGGFPLSGE
GGC
Additional BiKE-sialidase fusion sequences (BiKE sequence underlined;
linker italicized and bolded):
Anti-CD19 BiKE-sialidase fusion TAL3S
(SEQ ID NO: 25)
DIQLTQSPASLAVSLGQRATISCKASQSVDYDGDSYLNWYQQIPGQPPKLLIYDASN
LVSGIPPRFSGSGSGTDFTLNIHPVEKVDAATYHCQQSTEDPWTFGGGTKLEIKGGG
GSGGGGSGGGGSQVQLQQSGAELVRPGSSVKISCKASGYAFSSYWMNWVKQRPGQ
GLEWIGQIWPGDGDTNYNGKFKGKATLTADESSSTAYMQLSSLASEDSAVYFCARR
ETTTVGRYYYAMDYWGQGTTVTVSSGGGGSQVQLVQSGAEVKKPGESLKVSCKA
SGYTFTSYYMHWVRQAPGQGLEWMGIINPSGGSTSYAQKFQGRVTMTRDTSTSTV
YMELSSLRSEDTAVYYCARGSAYYYDFADYWGQGTLVTVSSGGGGSGGGGSGGG
GSSYVLTQPSSVSVAPGQTATISCGGHNIGSKNVHWYQQRPGQSPVLVIYQDNKRPS
GIPERFSGSNSGNTATLTISGTQAMDEADYYCQVWDNYSVLFGGGTKLTVLGGGGS
GGGGSGGGGS
MTENGMMNTNNTVCGANHDGAMSLAAPGDYGVACYRIPALAEAPNGWILAAFDA
RPHNCQDAPQANSIVQRISKDGGRSFEPQHVVAAGHDGVDKYGYSDPSYVVDRQT
GEVFLFFVKSYDAGFGTSQAGVDPSAREVLQAAVTSSIDNGVTWSEPRIITADITNSE
SWISRFASSGAGIQLTYGEHAGRLIQQYTIKELDGRYRAVSVFSDDHGATWHAGTP
VGDHMDENKVVELSDGRVMLNSRSSDGNGCRYVAISRDGGATYGPVIRETQLPDP
ENNAQIARAFPDAPEGSAQAKVLLYSSSSPSDRIDGLVRVSIDDGKTWSAGRRFTTG
PMAYSVIAALSHKAGGGYGLLYEGDNNNIMYTRISLDWLNGQLNVDGIGGFPLSGE
GGC
Anti-CD19 BiKE-sialidase fusion TAL4S
(SEQ ID NO: 26)
DIQLTQSPASLAVSLGQRATISCKASQSVDYDGDSYLNWYQQIPGQPPKLLIYDASN
LVSGIPPRFSGSGSGTDFTLNIHPVEKVDAATYHCQQSTEDPWTFGGGTKLEIKGGG
GSGGGGSGGGGSQVQLQQSGAELVRPGSSVKISCKASGYAFSSYWMNWVKQRPGQ
GLEWIGQIWPGDGDTNYNGKFKGKATLTADESSSTAYMQLSSLASEDSAVYFCARR
ETTTVGRYYYAMDYWGQGTTVTVSSGGGGSQVQLVQSGAEVKKPGESLKVSCKA
SGYTFTSYYMHWVRQAPGQGLEWMGIINPSGGSTSYAQKFQGRVTMTRDTSTSTV
YMELSSLRSEDTAVYYCARGSAYYYDFADYWGQGTLVTVSSGGGGSGGGGSGGG
GSSYVLTQPSSVSVAPGQTATISCGGHNIGSKNVHWYQQRPGQSPVLVIYQDNKRPS
GIPERFSGSNSGNTATLTISGTQAMDEADYYCQVWDNYSVLFGGGTKLTVLGGGGS
GGGGSGGGGSGGGGS
MTENGMMNTNNTVCGANHDGAMSLAAPGDYGVACYRIPALAEAPNGWILAAFDA
RPHNCQDAPQANSIVQRISKDGGRSFEPQHVVAAGHDGVDKYGYSDPSYVVDRQT
GEVFLFFVKSYDAGFGTSQAGVDPSAREVLQAAVTSSIDNGVTWSEPRIITADITNSE
SWISRFASSGAGIQLTYGEHAGRLIQQYTIKELDGRYRAVSVFSDDHGATWHAGTP
VGDHMDENKVVELSDGRVMLNSRSSDGNGCRYVAISRDGGATYGPVIRETQLPDP
ENNAQIARAFPDAPEGSAQAKVLLYSSSSPSDRIDGLVRVSIDDGKTWSAGRRFTTG
PMAYSVIAALSHKAGGGYGLLYEGDNNNIMYTRISLDWLNGQLNVDGIGGFPLSGE
GGC
Anti-EGFR BiKE-sialidase fusion TAL3S
(SEQ ID NO: 27)
QVQLQESGPGLVKPSETLSLTCTVSGGSVSSGSYYWSWIRQPPGKGLEWIGYIYYSG
STNYNPSLKSRVTISVDTSKNQFSLKLSSVTAADTAVYYCARNPISIPAFDIWGQGTM
VTVSSGGGGSGGGGSGGGGSQPVLTQPPSVSVAPGKTARITCGGNNIGSKSVHWYQ
QKPGQAPVLVIYYDSDRPSGIPERFSGSNSGNTATLTISRVEAGDEADYYCQVWDTS
SDHVLFGGGTKLTVLGGGGSQVQLVQSGAEVKKPGESLKVSCKASGYTFTSYYMH
WVRQAPGQGLEWMGIINPSGGSTSYAQKFQGRVTMTRDTSTSTVYMELSSLRSEDT
AVYYCARGSAYYYDFADYWGQGTLVTVSSGGGGSGGGGSGGGGSSYVLTQPSSVS
VAPGQTATISCGGHNIGSKNVHWYQQRPGQSPVLVIYQDNKRPSGIPERFSGSNSGN
TATLTISGTQAMDEADYYCQVWDNYSVLFGGGTKLTVLGGGGSGGGGSGGGGS
MTENGMMNTNNTVCGANHDGAMSLAAPGDYGVACYRIPALAEAPNGWILAAFDA
RPHNCQDAPQANSIVQRISKDGGRSFEPQHVVAAGHDGVDKYGYSDPSYVVDRQT
GEVFLFFVKSYDAGFGTSQAGVDPSAREVLQAAVTSSIDNGVTWSEPRIITADITNSE
SWISRFASSGAGIQLTYGEHAGRLIQQYTIKELDGRYRAVSVFSDDHGATWHAGTP
VGDHMDENKVVELSDGRVMLNSRSSDGNGCRYVAISRDGGATYGPVIRETQLPDP
ENNAQIARAFPDAPEGSAQAKVLLYSSSSPSDRIDGLVRVSIDDGKTWSAGRRFTTG
PMAYSVIAALSHKAGGGYGLLYEGDNNNIMYTRISLDWLNGQLNVDGIGGFPLSGE
GGC
Anti-EGFR BiKE-sialidase fusion TAL4S
(SEQ ID NO: 28)
QVQLQESGPGLVKPSETLSLTCTVSGGSVSSGSYYWSWIRQPPGKGLEWIGYIYYSG
STNYNPSLKSRVTISVDTSKNQFSLKLSSVTAADTAVYYCARNPISIPAFDIWGQGTM
VTVSSGGGGSGGGGSGGGGSQPVLTQPPSVSVAPGKTARITCGGNNIGSKSVHWYQ
QKPGQAPVLVIYYDSDRPSGIPERFSGSNSGNTATLTISRVEAGDEADYYCQVWDTS
SDHVLFGGGTKLTVLGGGGSQVQLVQSGAEVKKPGESLKVSCKASGYTFTSYYMH
WVRQAPGQGLEWMGIINPSGGSTSYAQKFQGRVTMTRDTSTSTVYMELSSLRSEDT
AVYYCARGSAYYYDFADYWGQGTLVTVSSGGGGSGGGGSGGGGS
SYVLTQPSSVSVAPGQTATISCGGHNIGSKNVHWYQQRPGQSPVLVIYQDNKRPSGI
PERFSGSNSGNTATLTISGTQAMDEADYYCQVWDNYSVLFGGGTKLTVLGGGGSG
GGGSGGGGSGGGGS
MTENGMMNTNNTVCGANHDGAMSLAAPGDYGVACYRIPALAEAPNGWILAAFDA
RPHNCQDAPQANSIVQRISKDGGRSFEPQHVVAAGHDGVDKYGYSDPSYVVDRQT
GEVFLFFVKSYDAGFGTSQAGVDPSAREVLQAAVTSSIDNGVTWSEPRIITADITNSE
SWISRFASSGAGIQLTYGEHAGRLIQQYTIKELDGRYRAVSVFSDDHGATWHAGTP
VGDHMDENKVVELSDGRVMLNSRSSDGNGCRYVAISRDGGATYGPVIRETQLPDP
ENNAQIARAFPDAPEGSAQAKVLLYSSSSPSDRIDGLVRVSIDDGKTWSAGRRFTTG
PMAYSVIAALSHKAGGGYGLLYEGDNNNIMYTRISLDWLNGQLNVDGIGGFPLSGE
GGC
7A5 scFv targeting Vγ9Vδ2 TCR (SEQ ID NO: 39):
EVQLQQSGAELARPGASVKLSCKASGFTFTDHYINWVKQRTGQGLEWIGQIYPGDG
NTYYNQKFKGKATLTADKSSSTAYMQLSSLTSEDSAVYFCAPNYGDYTIDFWGQG
TSVTVSSGGGGSGGGGSGGGGSSDIVMSQSPSSLAVSVGEKVTMSCKSSQSLLYSSN
QKNYLAWYQQKPGQSPKLLIYWASTRESGVPDRFTGSGSGTDFTLTISSVKAEDLA
VYYCQQYYRYHTFGTGTKLEIK
4D5-7A5 BiTE
(SEQ ID NO: 40)
HAMADIQMTQSPSSLSASVGDRVTITCRASQDVNTAVAWYQQKPGKAPKLLIYSAS
FLYSGVPSRFSGSRSGTDFTLTISSLQPEDFATYYCQQHYTTPPTFGQGTKVEIKGAS
GGGGSGGGGSGGGGSSEVQLVESGGGLVQPGGSLRLSCAASGFNIKDTYIHWVRQA
PGKGLEWVARIYPTNGYTRYADSVKGRFTISADTSKNTAYLQMNSLRAEDTAVYY
CSRWGGDGFYAMDYWGQGTLVTVSSGSGGGGSEVQLQQSGAELARPGASVKLSC
KASGFTFTDHYINWVKQRTGQGLEWIGQIYPGDGNTYYNQKFKGKATLTADKSSST
AYMQLSSLTSEDSAVYFCAPNYGDYTIDFWGQGTSVTVSSGGGGSGGGGSGGGGSS
DIVMSQSPSSLAVSVGEKVTMSCKSSQSLLYSSNQKNYLAWYQQKPGQSPKLLIYW
ASTRESGVPDRFTGSGSGTDFTLTISSVKAEDLAVYYCQQYYRYHTFGTGTKLEIK
4D5-7A5 BiTE-sialidase fusion (SEQ ID NO: 41):
HAMADIQMTQSPSSLSASVGDRVTITCRASQDVNTAVAWYQQKPGKAPKLLIYSAS
FLYSGVPSRFSGSRSGTDFTLTISSLQPEDFATYYCQQHYTTPPTFGQGTKVEIKGAS
GGGGSGGGGSGGGGSSEVQLVESGGGLVQPGGSLRLSCAASGFNIKDTYIHWVRQA
PGKGLEWVARIYPTNGYTRYADSVKGRFTISADTSKNTAYLQMNSLRAEDTAVYY
CSRWGGDGFYAMDYWGQGTLVTVSSGSGGGGSEVQLQQSGAELARPGASVKLSC
KASGFTFTDHYINWVKQRTGQGLEWIGQIYPGDGNTYYNQKFKGKATLTADKSSST
AYMQLSSLTSEDSAVYFCAPNYGDYTIDFWGQGTSVTVSSGGGGSGGGGSGGGGSS
DIVMSQSPSSLAVSVGEKVTMSCKSSQSLLYSSNQKNYLAWYQQKPGQSPKLLIYW
ASTRESGVPDRFTGSGSGTDFTLTISSVKAEDLAVYYCQQYYRYHTFGTGTKLEIKG
GGGSGGGGSMTENGMMNTNNTVCGANHDGAMSLAAPGDYGVACYRIPALAEAP
NGWILAAFDARPHNCQDAPQANSIVQRISKDGGRSFEPQHVVAAGHDGVDKYGYS
DPSYVVDRQTGEVFLFFVKSYDAGFGTSQAGVDPSAREVLQAAVTSSIDNGVTWSE
PRIITADITNSESWISRFASSGAGIQLTYGEHAGRLIQQYTIKELDGRYRAVSVFSDDH
GATWHAGTPVGDHMDENKVVELSDGRVMLNSRSSDGNGCRYVAISRDGGATYGP
VIRETQLPDPENNAQIARAFPDAPEGSAQAKVLLYSSSSPSDRIDGLVRVSIDDGKTW
SAGRRFTTGPMAYSVIAALSHKAGGGYGLLYEGDNNNIMYTRISLDWLNGQLNVD
GIGGFPLSGEGGC

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

We claim:

1. A fusion polypeptide comprising (a) a bispecific molecule and (b) a sialidase or enzymatic fragment thereof; wherein the bispecific molecule comprises two antibody moieties or antigen-binding fragments that respectively bind to an immune cell and an antigen associated with a disease.

2. The fusion polypeptide of claim 1, wherein the bispecific molecule comprises in tandem a first scFv that binds to the immune cell and a second scFv that binds to the antigen associated with a disease.

3. The fusion polypeptide of claim 2, wherein the bispecific molecule is a bispecific T cell engager (BiTE), and wherein the first scFv recognizes a T cell-specific molecule.

4. The fusion polypeptide of claim 3, wherein the T cell-specific molecule is CD3.

5. The fusion polypeptide of claim 3, wherein the BiTE selectively engages gammadelta T (γδT) cells, and wherein the T cell-specific molecule is Vγ9Vδ2 TCR.

6. The fusion polypeptide of claim 2, wherein the bispecific molecule is a bispecific innate cell engager, and wherein the first scFv recognizes a surface antigen on an innate immune cell.

7. The fusion polypeptide of claim 6, wherein the innate immune cell is NK cell or macrophage.

8. The fusion polypeptide of claim 7, wherein the surface antigen is CD16A or NKp44.

9. The fusion polypeptide of claim 2, wherein the disease is a tumor.

10. The fusion polypeptide of claim 9, wherein the second scFv binds to CD19, HER2 or PSMA.

11. The fusion polypeptide of claim 1, wherein the sialidase is a human sialidase, a viral sialidase or a bacterial sialidase.

12. The fusion polypeptide of claim 11, wherein the human sialidase is NEU1, NEU2, NEU3, NEU4 or isoform thereof.

13. The fusion polypeptide of claim 11, wherein the bacterial sialidase is B. infantis sialidase.

14. The fusion polypeptide of claim 1, wherein the sialidase is fused at the C-terminus or the N-terminus of the bispecific molecule.

15. The fusion polypeptide of claim 1, wherein the sialidase is fused to the bispecific molecule via a GS linker.

16. The fusion polypeptide of claim 15, wherein the GS linker comprises (GmS)n, wherein m is an integer from 1 to 6, and n is an integer from 1 to 10.

17. The fusion polypeptide of claim 15, wherein the GS linker comprises (GGGS)₂ (SEQ ID NO:2), (GGGGS)₂ (SEQ ID NO:29), (GGGGS)₃ (SEQ ID NO:30), or (GGGGS)₄ (SEQ ID NO:31).

18. The fusion polypeptide of claim 1, wherein the two antibody moieties or antigen-binding fragments are connected by a GS linker.

19. The fusion polypeptide of claim 1, wherein the bispecific molecule comprises a sequence that is at least 95% or 99% identical to the sequence set forth in any one of SEQ ID NOs: 6, 10, 12, 14, 31, 32 and 40.

20. The fusion polypeptide of claim 1, wherein the bispecific molecule comprises a sequence that is set forth in any one of SEQ ID NOs: 6, 10, 12, 14, 31, 32 and 40, or a conservatively modified variant thereof.

21. The fusion polypeptide of claim 1, comprising a sequence that is at least 95% or 99% identical to the sequence set forth in any one of SEQ ID NOs: 7, 8, 11, 13, 15, 23-28, and 41.

22. The fusion polypeptide of claim 1, comprising a sequence that is set forth in any one of SEQ ID NOs: 7, 8, 11, 13, 15, 23-28 and 41, or a conservatively modified variant thereof.

23. A pharmaceutical composition comprising a therapeutically effective amount of the fusion polypeptide of claim 1 and a pharmaceutically acceptable carrier.

24. A kit comprising the fusion polypeptide of claim 1.

25. A method for treating or ameliorating the symptoms of a disease or disorder in a subject, comprising administering to the subject a pharmaceutical composition comprising the fusion polypeptide of claim 1.

26. The method of claim 25, wherein the disease is a tumor.

27. A polynucleotide encoding the fusion polypeptide of claim 1.

28. A vector harboring the polynucleotide of claim 27.

29. A lipid nanoparticle (LNP) that is formulated with the polynucleotide of claim 27.


SEQUENCE LISTING (41 sequences)

SEQ ID NO: 1 - protein, 4 aa, synthetic construct
GGGS

SEQ ID NO: 2 - protein, 8 aa, synthetic construct
GGGSGGGS

SEQ ID NO: 3 - protein, 252 aa, synthetic construct
HAMADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGASGGGGSGGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGS

SEQ ID NO: 4 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 5 - protein, 394 aa, Bifidobacterium longum
MTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC

SEQ ID NO: 6 - protein, 495 aa, synthetic construct
HAMADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGASGGGGSGGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 7 - protein, 897 aa, synthetic construct
HAMADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGASGGGGSGGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGSGGGSMTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC

SEQ ID NO: 8 - protein, 897 aa, synthetic construct
MTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGCGGGSGGGSHAMADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGASGGGGSGGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 9 - protein, 237 aa, synthetic construct
EVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSGGGGSGGGGSGGGGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLK

SEQ ID NO: 10 - protein, 484 aa, synthetic construct
EVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSGGGGSGGGGSGGGGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 11 - protein, 886 aa, synthetic construct
EVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSGGGGSGGGGSGGGGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGSGGGSMTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC

SEQ ID NO: 12 - protein, 498 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 13 - protein, 900 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGSGGGSMTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC

SEQ ID NO: 14 - protein, 491 aa, synthetic construct
QVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSDIVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELKGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 15 - protein, 893 aa, synthetic construct
QVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSDIVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELKGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGSGGGSMTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC

SEQ ID NO: 16 - protein, 415 aa, Homo sapiens
MTGERPSTALPDRRWGPRILGFWGGCRVWVFAAIFLLLSLAASWSKAENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISVYGTL

SEQ ID NO: 17 - protein, 380 aa, Homo sapiens
MASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 18 - protein, 428 aa, Homo sapiens
MEEVTTCSFNSPLFRQEDDRGITYRIPALLYIPPTHTFLAFAEKRSTRRDEDALHLVLRRGLRIGQLVQWGPLKPLMEATLPGHRTMNPCPVWEQKSGCVFLFFICVRGHVTERQQIVSGRNAARLCFIYSQDAGCSWSEVRDLTEEVIGSELKHWATFAVGPGHGIQLQSGRLVIPAYTYYIPSWFFCFQLPCKTRPHSLMIYSDDLGVTWHHGRLIRPMVTVECEVAEVTGRAGHPVLYCSARTPNRCRAEALSTDHGEGFQRLALSRQLCEPPHGCQGSVVSFRPLEIPHRCQDSSSKDAPTIQQSSPGSSLRLEEEAGTPSESWLLYSHPTSRKQRVDLGIYLNQTPLEAACWSRPWILHCGPCGYSDLAALEEEGLFGCLFECGTKQECEQIAFRLFTHREILSHLQGDCTSPGRNPSQFKSN

SEQ ID NO: 19 - protein, 461 aa, Homo sapiens
MRPADLPPRPMEESPASSSAPTETEEPGSSAEVMEEVTTCSFNSPLFRQEDDRGITYRIPALLYIPPTHTFLAFAEKRSTRRDEDALHLVLRRGLRIGQLVQWGPLKPLMEATLPGHRTMNPCPVWEQKSGCVFLFFICVRGHVTERQQIVSGRNAARLCFIYSQDAGCSWSEVRDLTEEVIGSELKHWATFAVGPGHGIQLQSGRLVIPAYTYYIPSWFFCFQLPCKTRPHSLMIYSDDLGVTWHHGRLIRPMVTVECEVAEVTGRAGHPVLYCSARTPNRCRAEALSTDHGEGFQRLALSRQLCEPPHGCQGSVVSFRPLEIPHRCQDSSSKDAPTIQQSSPGSSLRLEEEAGTPSESWLLYSHPTSRKQRVDLGIYLNQTPLEAACWSRPWILHCGPCGYSDLAALEEEGLFGCLFECGTKQECEQIAFRLFTHREILSHLQGDCTSPGRNPSQFKSN

SEQ ID NO: 20 - protein, 496 aa, Homo sp.
MMSSAAFPRWLSMGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 21 - protein, 497 aa, Homo sapiens
MMSSAAFPRWLQSMGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 22 - protein, 484 aa, Homo sapiens
MGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 23 - protein, 900 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSQVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSGGGGSGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGGGSGGGGSMTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC

SEQ ID NO: 24 - protein, 893 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSGGGGSGGGGSGGGGSQPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVLGGGGSQVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSGGGGSGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGGGSGGGGSMTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC

SEQ ID NO: 25 - protein, 905 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSQVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSGGGGSGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGGGSGGGGSGGGGSMTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC

SEQ ID NO: 26 - protein, 910 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSQVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSGGGGSGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGGGSGGGGSGGGGSGGGGSMTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC

SEQ ID NO: 27 - protein, 898 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSGGGGSGGGGSGGGGSQPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVLGGGGSQVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSGGGGSGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGGGSGGGGSGGGGSMTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC

SEQ ID NO: 28 - protein, 903 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSGGGGSGGGGSGGGGSQPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVLGGGGSQVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSGGGGSGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGGGSGGGGSGGGGSGGGGSMTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC

SEQ ID NO: 29 - protein, 10 aa, synthetic construct
GGGGSGGGGS

SEQ ID NO: 30 - protein, 15 aa, synthetic construct
GGGGSGGGGSGGGGS

SEQ ID NO: 31 - protein, 20 aa, synthetic construct
GGGGSGGGGSGGGGSGGGGS

SEQ ID NO: 32 - protein, 496 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSQVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSGGGGSGGGGSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 33 - protein, 368 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSGGGGSGGGGSGGGGSQPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVLGGGGSQVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 34 - protein, 5 aa, synthetic construct
SGGGG

SEQ ID NO: 35 - protein, 10 aa, synthetic construct
SGGGGSGGGG

SEQ ID NO: 36 - protein, 15 aa, synthetic construct
SGGGGSGGGGSGGGG

SEQ ID NO: 37 - protein, 20 aa, synthetic construct
SGGGGSGGGGSGGGGSGGGG

SEQ ID NO: 38 - protein, 5 aa, synthetic construct
GGGGS

SEQ ID NO: 39 - protein, 246 aa, synthetic construct
EVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTIDFWGQGTSVTVSSGGGGSGGGGSGGGGSSDIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYHTFGTGTKLEIK

SEQ ID NO: 40 - protein, 503 aa, synthetic construct
HAMADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGASGGGGSGGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSGGGGSEVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTIDFWGQGTSVTVSSGGGGSGGGGSGGGGSSDIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYHTFGTGTKLEIK

SEQ ID NO: 41 - protein, 907 aa, synthetic construct
HAMADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGASGGGGSGGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSGGGGSEVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTIDFWGQGTSVTVSSGGGGSGGGGSGGGGSSDIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYHTFGTGTKLEIKGGGGSGGGGSMTENGMMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSAREVLQAAVTSSIDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC